(19) United States
(12) Patent Application Publication
(10) Pub. No.: US 2026/0061091 A1
(43) Pub. Date: Mar. 5, 2026

(54) MUSSEL-INSPIRED TISSUE ADHESIVES AND METHODS OF USE THEREOF

(71) Applicant: Purdue Research Foundation, West Lafayette, IN (US)

(72) Inventors: Jonathan Wilker, West Lafayette, IN (US); Julie Liu, West Lafayette, IN (US); Gudrun Schmidt, West Lafayette, IN (US); Aishwarya Menon, Lafayette, IN (US)

(21) Appl. No.: 19/105,023

(22) PCT Filed: Aug. 21, 2023
(86) PCT No.: PCT/US2023/072601

§ 371 Date: Feb. 19, 2025

Related U.S. Application Data
(60) Provisional application No. 63/399,522, filed on Aug. 19, 2022.

(51) Int. Cl. A61L 24/10; A61L 24/00

(52) U.S. Cl. CPC A61L 24/108; A61L 24/001; A61L 2430/20; A61L 2430/22; A61L 2430/28; A61L 2430/34

(57) ABSTRACT

Bioinspired adhesives comprising a macromolecule such as zein and one or more catechol- or gallol-containing compounds are provided. The adhesive can also comprise an iron crosslinker, an interfacial crosslinker, or both an iron crosslinker and an interfacial crosslinker. Methods of manufacturing and using the adhesive are also provided.

### PRIORITY

This application is related to and claims the priority benefit of U.S. Provisional Patent Application No. 63/399,522 filed Aug. 19, 2022. The content of the aforementioned application is hereby incorporated by reference in its entirety into this disclosure.

### TECHNICAL FIELD

The present disclosure relates to a bioinspired adhesive comprising zein and one or more catechol- or gallol-containing compounds that imparts superior adhesive properties and is biocompatible. The adhesive can also comprise an iron crosslinker, an interfacial crosslinker, or both an iron crosslinker and an interfacial crosslinker. Methods of manufacturing and using the adhesive are also provided.

### BACKGROUND

Leaking wounds, either in the form of bleeding or through release of bodily fluids and gases, are a major complication in most surgeries across the world. Traditionally, such wounds were sealed using sutures, staples, and wires; however, these traditional wound closure methods are increasingly being replaced by tissue sealants or adhesives as they are invasive and can lead to additional complications that increase the risk of lengthy wound closure times, tissue damage, infection and incomplete wound closure. According to the Surgical Sealants and Adhesives Global Market Report 2022 published by ReportLinker, the global market for surgical sealants and adhesives is expected to grow from $2.12 billion in 2021 to $2.39 billion in 2022 at a compound annual growth rate (CAGR) of 13%. Moreover, the market is expected to grow to $3.13 billion by 2026 at a CAGR of 6.9%.

Two key requirements of an effective tissue sealant are (1) ability to bond with tissues in the presence of body fluids and under pressurized conditions (at least until tissue regeneration), and (2) being cyto-compatible with minimal (if any) toxicity or inducement of a foreign body reaction in the subject (including, for example, being able to degrade within the body into non-toxic byproducts). Two of the first sealants to be commercialized for surgical applications were fibrin-based and cyanoacrylate (butyl and octyl cyanoacrylate)-based sealants. Fibrin sealant (Tisseel, Vistaseal™, etc.) is a two-component sealant consisting of fibrinogen and thrombin which, when applied on a targeted site, react together and mimic a blood clot to seal the wounds. Though these sealants showed initial promise, they exhibited poor adhesion strengths and posed a risk of disease transmission (due to their source being pooled donor blood).

Cyanoacrylate adhesives (Trufill®. Omnex, etc.) in contrast exhibited fast gelation times and instant tissue sealing properties. However, cyanoacrylates break down within the body to toxic byproducts including formaldehyde and exhibited mechanical mismatch with soft tissues which led them to be used strictly for topical adhesion applications only. These limitations of the earliest commercially available tissue sealant have led to the emergence of newer and improved sealants based on albumin and glutaraldehyde (BioGlue®, PreveLeak, etc.), polyethylene glycol (Progel™, Coseal etc.) and polyurethanes (TissuGlu©). Although, these newer adhesives have shown promise, there is still a need for an effective alternative that does not exhibit toxicity and poor adhesion concerns and is cost-effective to procure.

Nature and bioinspired materials have also gained a lot of traction in recent years. In this regard, adhesives inspired from sea-creatures like mussels, barnacles, and oysters have been researched as a source of water-resistant adhesives. These sea-creatures can adhere tightly to surfaces underwater, while withstanding not only a wet and changing sea environment, but also turbulent waves. Further, such marine life can remain adhered to rocks for years at a time. These characteristics have inspired researchers to develop tissue adhesives and sealants that mimic their adhesion chemistry.

The attachment system of sea mussels is called “byssus” which is secreted from the foot of the mussel and consists of several threads terminating in an adhesive plaque. The byssus is composed of roughly 25-30 different proteins out of which only 7-8 are present in the plaque and only 5 (Mytilus foot protein type 2 (mfp-2), Mytilus foot protein type 3 (mfp-3), Mytilus foot protein type 4 (mfp-4), Mytilus foot protein type 5 (mfp-5) and Mytilus foot protein type 6 (mfp-6)) are unique to the plaque. One of the key components in sea mussel adhesive is a molecule called 1-3,4-dihydroxyphenylalanine (DOPA), which is largely found in mfp-3 and mfp-5.

Mussel-inspired adhesives have been synthesized using a variety of strategies. Natural polysaccharides like chitin, cellulose, hyaluronic acid, chitosan, silk fibroin, and the like have been modified with 3,4-dihydroxy-L-phenylalanine (L-DOPA), hydro caffeic acid, dopamine by surface chemistry and covalent conjugate to develop mussel-inspired, polysaccharide-based tissue adhesives. The polymer chain backbone of synthetic polymers has also been modified using dopamine or L-DOPA. Synthetic polymers like polystyrene, polylactic acid, and polyethylene glycol have been tuned to incorporate DOPA chemistry. Moreover, DOPA groups have been incorporated into recombinant protein systems via lysine and tyrosine incorporation. However, these conventional mussel-inspired adhesives can be costly and difficult to synthesize, require curing additives and other components, exhibit poor adhesivity, and can exhibit toxicity issues.

A need remains for a natural, biodegradable, easily synthesizable, scalable, and low-cost adhesive that is biocompatible, exhibits strong adhesion (even when subjected to biofluids), and has a short cure time.

### SUMMARY

Adhesives are provided. An adhesive can comprise zein and one or more catechol- or gallol-containing compounds. Optionally, the zein is present in at or about 25-45 weight percent (wt. %) (such as 25-45 wt. %) of the adhesive prior to curing. In certain embodiments, the adhesive further comprises an interfacial crosslinker, an iron crosslinker, or both an iron crosslinker and an interfacial crosslinker. The one or more catechol- or gallol-containing compounds can comprise catechol, vanillin, caffeic acid, juglone (5-hydroxy-1,4-naphthoquinone), catecholamine, resveratrol, 3,4-dihyroxyphenylacetic acid (DOPAC), catechin hydrate, 3,4-dihydroxy benzoic acid, 3,4-dihydroxybenzaldehyde, tannic acid (TA), gallic acid, gallotannins, ellagic acid, eugallol, 1,2,3-trihydroxybenzene, quercetin, gallol, or any combination thereof. The adhesive can be formulated at or around a neutral pH (about pH=7). The adhesive can be formulated at a pH of at or between about 6-8 (e.g., a pH of about 5.5, a pH of about 6, a pH of about 6.5, a pH of about 7, a pH of about 7.5, a pH of about 8, or a pH of about 8.5).

Where the adhesive comprises an iron crosslinker, the iron crosslinker can comprise ferrous sulfate (FeSO₄), ferric chloride (FeCl₃), an iron oxide nanoparticle, ferric nitrate (Fe(NO₃)₃), iron(III) acetylacetonate (Fe(acac)₃ or Fe(C₅H₇O₂)₃), potassium ferrate (K₂FeO₄), or any combination thereof. Where the adhesive comprises an interfacial crosslinker, the interfacial crosslinker adhesive comprises an interfacial crosslinker can comprise transglutaminase (TGnase) (including without limitation microbial TGnase (mTG)), ethyl-3-(3-dimethylaminopropyl)carbodiimide hydrochloride (EDC), N-hydroxy succinimide (NHS), an elastin-like polypeptide (ELP), polyethylene glycol (PEG), a dendrimer, glutaraldehyde, and/or photocrosslinkers. In certain embodiments, the interfacial crosslinker comprises TA. In certain embodiments, zein is about 25-45 wt. % (such as 25-45 wt. %) and the one or more catechol- or gallol-containing compounds is TA present in at or about 5-30 wt. % (such as 5-30 wt. %), prior to curing the adhesive. In certain embodiments, zein is at or about 40 weight percent (wt. %) and the one or more catechol- or gallol-containing compounds is TA present in at or about 7 wt. % of the uncured adhesive. In certain embodiments, zein is at or about 26 wt. % and the one or more catechol- or gallol-containing compounds is TA present in at or about 21 wt. % of the uncured adhesive. In certain embodiments, zein is at or about 26 wt. % and the one or more catechol- or gallol-containing compounds is TA present in at or about 28 wt. % of the uncured adhesive. In certain embodiments, zein is at or about 30 wt. % and the one or more catechol- or gallol-containing compounds is TA present in at or about 21 wt. % of the uncured adhesive.

The adhesive can further comprise a solvent. In certain embodiments, the solvent comprises an organic solvent. In certain embodiments, the solvent comprises an aqueous solvent. In certain embodiments, the solvent is both an aqueous and organic solvent. The solvent can comprise ethanol and water. The ethanol and water can be present by volume in at or about a ratio of (2-5):1. The ethanol and water can be present in at or about a 5:1 ratio, a 4:1 ratio, a 3:1 ratio, or a 2:1 ratio.

The adhesive can have a tensile strength of up to about 20 MPa.

The adhesive can further comprise at least one oxidant. The adhesive can further comprise at least one filler. The at least one filler can comprise hydroxyapatite, calcium carbonate, calcium phosphate, magnesium phosphate, laponite, montmorillonite, or a combination of any of the foregoing.

Tissue sealants are also provided. In certain embodiments, the tissue sealant comprises an adhesive hereof. In certain embodiments, the tissue sealant comprises zein, TA, and a solvent. The solvent can comprise an aqueous solvent, an organic solvent, or both an aqueous and organic solvent. In certain embodiments, the solvent comprises ethanol and water. In certain embodiments, the tissue sealant consists of (e.g., only) zein, TA, ethanol and water.

The tissue sealant can further comprise an interfacial crosslinker, an iron crosslinker, or both an interfacial crosslinker and an iron crosslinker. In certain embodiments, the interfacial crosslinker comprises TGnase. The TGnase can be combined with the zein, TA and solvent, or optionally applied to a targeted tissue sequentially with the tissue sealant.

In certain embodiments, the tissue sealant further comprises at least one filler. The at least one filler can comprise hydroxyapatite, calcium carbonate, calcium phosphate, magnesium phosphate, laponite, montmorillonite, or a combination of any of the foregoing.

Methods of sealing a damaged tissue of a subject (e.g., a mammal) are also provided. In certain embodiments, the method comprises applying an adhesive or tissue sealant described herein to a damaged tissue. The method can further comprise applying an interfacial crosslinker to the damaged tissue to form a first layer and applying the adhesive or tissue sealant to the first layer (e.g., injected onto and/or spread over the damaged tissue).

The interfacial crosslinker can comprise a viscous solution of TGnase. In certain embodiments, the viscous solution of TGnase comprises TGnase and water. The interfacial crosslinker can comprise a Tgnase powder (e.g., spread or sprinkled over the damaged tissue).

In certain embodiments, the method comprises forming the first layer and applying the adhesive to the first layer.

The damaged tissue can be selected from the group consisting of skin, heart tissue, stomach tissue, lung tissue, liver tissue, intestines, dura mater, and an aorta. In certain embodiments, the subject is a mammal. In certain embodiments, the subject is a human.

The method can further comprise allowing the adhesive or tissue sealant to cure. The adhesive or tissue sealant can cure in the presence of a biofluid. The damaged tissue can be in an oral environment. In certain embodiments, where the damaged tissue is in an oral environment, the adhesive or tissue sealant is allowed to cure in saliva.

The one or more catechol- or gallol-containing compounds of the adhesive can interact with functional groups of the damaged tissue to facilitate an adhesive bond with the damaged tissue.

The method can further comprise applying a suture to the damaged tissue. The method can further comprise applying a graft to the damaged tissue. In certain embodiments, the adhesive or tissue sealant is applied to the damaged tissue and the graft is thereafter applied to the damaged tissue and adhesive or tissue sealant. In certain embodiments, the graft is applied to the damaged tissue and thereafter, the adhesive or tissue sealant is applied over the surface of the graft and onto tissue surrounding the graft.

The method can further comprise curing the adhesive by removing or facilitating removal of the solvent from the adhesive. Curing the adhesive can comprise exposing the adhesive or tissue sealant to oxygen or heat. The adhesive or tissue sealant can be applied to the graft and allowed to cure or cured for less than about 15 minutes (such as less than 15 minutes) before applying the adhesive/tissue sealant and graft to the damaged tissue. The adhesive or tissue sealant can be applied to the graft and allowed to cure for less than about 40 minutes (such as less than 40 minutes) before applying the adhesive/tissue sealant and graft to the damaged tissue.

The adhesive or tissue sealant applied can be about 80 μl to 150 μl in volume. The adhesive or tissue sealant applied can be about 10 μl to 25 ml in volume. The adhesive or tissue sealant applied can be at or about 150 μl in volume.

Methods of formulating an adhesive are provided. A method of formulating an adhesive can comprise forming a first solution by combining zein, one or more catechol- or gallol-containing compounds, a solvent, and, optionally, an interfacial crosslinker, an iron crosslinker, or both an iron crosslinker and an interfacial crosslinker; adjusting a pH of the first solution to at or about 6-8 (e.g., such as 6-8) if needed; and curing the first solution to substantially remove the solvent therefrom.

Curing the first solution can comprise incubating the first solution at or between about 31.0° C. to about 42.0° C. Curing the first solution can comprise incubating the first solution at or about 37° C. Curing the first solution can comprise exposing the first solution to oxygen. Curing the first solution can comprise light curing.

The method can further comprise applying the first solution (e.g., the incubated first solution) to a layer comprising TGnase. In certain embodiments, the TGnase is a powder. In certain embodiments, the TGnase is a viscous solution comprising TGnase and water. In certain embodiments, the first solution further comprises TGnase.

The one or more catechol- or gallol-containing compounds can be selected from the group consisting of TA, vanillin, gallic acid, caffeic acid, juglone, quercetin, catechin hydrate, 3,4-dihydroxy-benzoic acid, and 3,4-dihydroxybenzaldehyde.

The solvent can comprise ethanol and water. In certain embodiments, the ethanol to water ratio of the solvent of the first solution can be about 5:1, about 4:1, about 3:1, or about 2:1. The one or more catechol- or gallol-containing compounds of the first solution can comprise TA, and the first solution can comprise at or about 40 wt. % zein and at or about 7 wt. % TA. The one or more catechol- or gallol-containing compound of the first solution can comprise TA, and the first solution can comprise at or about 26 wt. % zein and at or about 21 wt. % TA. The one or more catechol- or gallol-containing compound of the first solution can comprise TA, and the first solution can comprise at or about 26 wt. % zein and at or about 28 wt. % TA. The one or more catechol- or gallol-containing compound of the first solution can comprise TA, and the first solution can comprise at or about 30 wt. % zein and at or about 21 wt. % TA.

In certain embodiments, the amount of one or more of the zein, the one or more catechol- or gallol-containing compounds, and the solvent of the first solution can be adjusted to tune one or more characteristics of the adhesive (e.g., modulus, stiffness, etc.) to correspond with mechanical properties of a targeted tissue.

Kits for preparing an adhesive or a tissue sealant hereof are also provided. The kit can comprise at least one of the adhesives or tissue sealants hereof, and instructions for use. In certain embodiments, the kit comprises zein, one or more catechol- or gallol-containing compounds, and a solvent, wherein the zein and one or more catechol- or gallol-containing compounds are hydrated by the solvent for immediate use. In certain embodiments, the kit comprises zein, one or more catechol- or gallol-containing compounds, and a solvent, wherein the zein is hydrated by the solvent and stored in a first container and the one or more catechol- or gallol-containing compounds are stored in a second container (separated from the first container).

### BRIEF DESCRIPTION OF DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1 shows a schematic illustration of burst pressure test set up as used in Example 3.

FIG. 2 shows a schematic illustration of a test set up for determining long term adhesion and stability of the sealant.

FIG. 3 shows a graph of data related to the effect of ethanol to water ratio on lap shear strength of Z40T7.

FIG. 4 shows a graph of data related to the overall solvent concentration on burst pressure of zein-based sealant Z40T7.

FIGS. 5A-5C show graphical data related to how the ratio of zein and tannic acid (TA) concentrations in various sealant formulation affects the final burst pressure strengths of the sealants. The amount of TA varied was limited by the final viscosity and consistency of the final formulation. FIG. 5A shows data related to the effect of varying TA concentration from 1-14 wt. % at 40 wt. % zein. FIG. 5B shows data related to the effect of varying TA concentration from 7-21 wt. % at 30 wt. % zein. FIG. 5C shows data related to the effect of varying TA concentration from 7-28 wt. % at 26 wt. % zein.

FIGS. 6A and 6B show graphical data related to the effect of the addition of Fe crosslinker on burst pressure strength Z40T7 and Z26T21, respectively.

FIGS. 7A-7D show graphical data related to the effect of pH on the burst pressure strength of the top 4 sealant formulations (i.e., those that exhibited the highest burst pressure values), with each formula prepared at various pH values (pH=5, 7, and 9), FIG. 7A showing data for Z40T7, FIG. 7B showing data for Z26T21, FIG. 7C showing data for Z40T7±0.5 wt. % Fe, and FIG. 7D showing data for Z26T21+0.1 wt. % Fe.

FIGS. 8A-8C show graphical data related to the effect on burst pressure of Z40T7 and Z26T21 with the inclusion of three different interfacial crosslinkers: Chitosan (Chi), EDC/NHS and Transglutaminase (TGnase), with FIG. 8A showing the formulation with Chitosan as the cross-linker, FIG. 8B showing the formulation with EDC/NHS as the cross-linker, and FIG. 8C showing the formulation with transglutaminase as the cross-linker.

FIG. 9 shows a graph of the burst pressure on porcine skin tissues of the zein-based sealants, Tisseel and Superglue, all at 30 minutes and 2-hour cure times as compared with the normal abdominal pressure on skin.

FIG. 10 shows a graph of the burst pressure on porcine sausage casing (processed intestine) of the zein-based sealants, Tisseel and Superglue, all at 30 minutes and 2-hour cure times as compared with the normal physiological pressure in the sausage casing.

FIG. 11 shows a graph of the burst pressure on porcine heart tissues of the zein-based sealants, Tisseel, and Superglue, all at 30-minute and 2-hour cure times as compared with the normal physiological pressure in the heart which is approximately the normal blood pressure.

FIG. 12 shows a graph of the burst pressure on porcine stomach tissues of all the zein-based sealants, Tisseel, and Superglue, all at 30-minute and 2-hour cure times as compared with the normal physiological pressure in the stomach.

FIG. 13 shows a graph of the burst pressure on porcine lung tissue of all the zein-based sealants, Tisseel, and Superglue, all at 30-minute and 2-hour cure times as compared with the normal physiological pressure in the lungs.

FIG. 14 shows a graph of the burst pressure on porcine liver tissue of all the zein-based sealants, Tisseel, and Superglue, all at 30-minute and 2-hour cure times as compared with the normal physiological pressure in the liver.

FIG. 15 shows a graph of the burst pressure on porcine aorta tissue of all the zein-based sealants, Tisseel, and Superglue, all at 30-minute and 2-hour cure times as compared with the normal physiological pressure in the aorta which is approximately the normal blood pressure.

FIGS. 16A and 16B show graphs of the burst pressure on porcine dura tissue (e.g., dura mater) of all zein-based sealants and Tisseel at 30-minute and 2-hour cure times as compared with the normal physiological pressure in the dura.

FIG. 17 shows a graph of lap shear strength of all zein-based adhesives at 30-minute and 2-hour cure times.

FIGS. 18A-18C shows images of steps of an ex vivo model of stomach tissue showing dyed water leaking from a 3 mm puncture in the stomach (FIG. 18A), sealing the puncture underwater with adhesive Z40T7 (FIG. 18B), and instant sealing of the puncture following application of the adhesive Z40T7 underwater puncture with no leaks (FIG. 18C).

FIGS. 19A-19C are images of steps of an ex vivo flow model of sausage casing showing water leaking from a 3 mm puncture in sausage casing with continuous flow (FIG. 19A), sealing the puncture with Z40T7 (FIG. 19B), and the puncture being sealed within 10 seconds of adhesive Z40T7 application with no more leaks (FIG. 19C).

FIGS. 20A and 20B relate to a study designed to assess the ability of the sealants to remain adhered on the tissues in wet environments for extended periods of time (up to 72 hours), with FIG. 20A showing images of the casings tested with (i) Z40T7, (ii) Z40T7+Tgnase, (iii) Z26T21, and (iv) Z26T21+Tgnase, and FIG. 20B showing graphical data related to the amount of permeated PBS collected after 72 hours that leaked from the casings shown in FIG. 20A.

FIGS. 21A and 21B are images of a 3 mm puncture on the dorsal section of a rat cadaver (FIG. 21A) and a puncture sealed with Z40T7 (FIG. 21B).

FIG. 22 shows a graph of the burst pressure of all zein-based sealants and Tisseel on porcine small intestine tissues at 30-minute and 2-hour cure times compared with the normal physiological pressure in the small intestine.

FIG. 23 is a graph of the lap shear strength of various zein and TA ratios.

FIG. 24 is a graph of the lap shear strength of the identified formulations of FIG. 23 compared against ARTISS at a 2-hour cure time.

FIGS. 25A-25C show results of a live/dead assay related to cytocompatibility for cells exposed to leachate at 1×, 10×, and 100× dilutions, with FIG. 25A showing a graph of the results, FIG. 25B showing images of the results, and FIG. 25C showing PrestroBlue metabolism results.

FIG. 26 is a graph of the burst pressure of Z40T7 and Z26T21 on sausage casing before and after gamma irradiation at 30 minutes cure time.

FIG. 27 is images of wound healing for the 7-day cohort at days 0, 4, and 7, following application of Z40T7, Z40T7+suture, Tisseel, and sutures only.

FIG. 28 is images of wound healing for the 14-day cohort at days 0, 4, 7, 10, and 14 following application of Z40T7, Z40T7+suture, Tisseel, and sutures only.

FIG. 29 shows burst pressure values of zein adhesives on sausage casing with collagen casing as a graft, at 30-minute cure time.

FIG. 30 shows burst pressure values of zein adhesives on sausage casing with collagen casing as a graft, at 15 minutes cure time (adhesive applied all over the graft material).

FIG. 31 shows burst pressure values of Z40T7+TGnase on dura mater with a graft compared against a sausage casing with a collagen casing graft, at 15-minute cure time (adhesives applied all over the graft material).

FIG. 32 shows burst pressures of Z26T21 when applied directly either on dura mater or a graft.

FIG. 33 a graph that shows the effect of increasing adhesive volume when applied on dura in combination with a graft at a 15-minute cure time.

FIG. 34 shows a graph of data related to the effect of transglutaminase on burst pressure when adhesive was applied directly on dura.

FIG. 35 is a graph of data related to the effect of cure time on burst pressure of an adhesive.

FIG. 36 shows comparative graphs of data related to the effect of puncture size on burst pressure.

FIG. 37 is a graph of data related to lap shear adhesion strength of dura when bonded to a graft.

FIG. 38 shows images of the amount of adhesive remaining on hydroxyapatite (HAP) substrate discs for Z40T7 and Z26T21 when studied in the presence of biofilm and PBS at two different pressure settings.

FIG. 39 shows the effect of staining time on bare HAP substrates (control).

FIG. 40 shows images of the effect of hydrochloric acid (HCl) cleaning on adhesion on HAP discs.

FIG. 41 are graphs of data related to the effect of curing in air versus curing under saliva for the zein adhesives hereof coated on limestone substrates.

FIG. 42 shows images of the effect of thin coating versus thick coating for zein adhesives on HAP-coated glass substrates.

FIG. 43 shows images of the effect of HAP powder incorporation as filler in Z26T21 on adhesion on HAP-coated glass substrates.

While the present disclosure is susceptible to various modifications and alternative forms, exemplary embodiments thereof are shown by way of example in the drawings and are herein described in detail.

While the present disclosure is susceptible to various modifications and alternative forms, exemplary embodiments thereof are shown by way of example in the drawings and are herein described in detail.

### DETAILED DESCRIPTION

While the concepts of the present disclosure are illustrated and described in detail in the description herein, results in the description are to be considered as exemplary and not restrictive in character; it being understood that only the illustrative embodiments are shown and described, and that all changes and modifications that come within the spirit of the disclosure are desired to be protected.

The present disclosure generally relates to a completely bio-based, biodegradable adhesive or tissue sealant that can be easily synthesized, is scalable, and low-cost to produce. Aspects of the adhesive are sea-mussel inspired; the adhesive can be based on a macromolecule (e.g., a protein or polysaccharide) and at least one catechol- or gallol-containing compound. As used herein, the terms “adhesive” and “sealant” are used interchangeably unless otherwise indicated. Optionally, the macromolecule is present in at or about 25-45 weight percent (wt. %) of the adhesive prior to curing (inclusive of the end points of the specified range).

The macromolecule can be a protein, a polysaccharide, a synthetic polymer, a nucleic acid, or any other macromolecule to the extent it is capable of crosslinking with the catechol- or gallol-containing compound. In certain embodiments, the macromolecule is a corn protein. In certain embodiments, the macromolecule is zein. Zein is an alcohol-soluble prolamine protein present in corn or maize. Pure zein can be clear, non-toxic, and water-insoluble. Zein is a natural degradable material, with a strong-hydrophobicity. The amino acid sequence of naturally-occurring (i.e., native) zein is publicly available.

Zein proteins can include, without limitation, α-zeins (e.g., having a molecular weight ranging from about 20 to about 30 kilodaltons (kd), β-zeins (e.g., having a molecular weight ranging from about 18 to 22 kd), γ-zeins (e.g., having a molecular weight ranging from about 25 to 35 kd), δ-zeins, and/or variants of any of the foregoing (i.e. modified variant). In certain embodiments, the zein can be native (unmodified) zein. In certain embodiments, the zein has a molecular weight of about 15 kd.

In certain embodiments, the zein can be a modified variant. “Modified variant” as used herein means zein proteins having an amino acid sequence that is not naturally occurring, behave similarly to native zeins, and are soluble in alcohol. In certain embodiments, a modified variant can have about 80%, about 85%, about 90%, about 91%, about 92%, about 93%, about 94%, about 95%, about 96%, about 97%, about 98%, about 99%, or about 99.5% sequence identity with the amino acid sequence of an amino acid sequence of naturally occurring (i.e., native or wild type) zein.

“Percent (%) sequence identity” with respect to a reference to an amino acid or polypeptide sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys online), and alignments can be done using, for example, the ClustalW algorithm (VNTI software, InforMax Inc.). Further, a sequence database can be searched using the nucleic acid or amino acid sequence of interest. Algorithms for database searching are typically based on the BLAST software (Altschul et al., 1990), but those skilled in the art can determine appropriate parameters for aligning sequences, including any algorithms needed to achieve maximal alignment over the full length of the sequences being compared. In some embodiments, the percent identity can be determined along the full-length of the nucleic acid or amino acid sequence. Means for modifying a zein structural gene are well known in the art and can be employed according to well-known and understood principles in the art to achieve the desired modification.

The catechol- or gallol-containing compound can be any compound comprising one or more catechol or gallol moieties. A catechol comprises a chemical structure with two adjacent hydroxyl (—OH) groups on a benzene ring. A catechol moiety can comprise the structure:

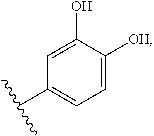 [SMILES: *c1ccc(O)c(O)c1]

wherein the

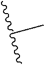

is the point of attachment to the compound. A gallol (or pyrogallol) moiety comprises a chemical structure with three hydroxyl (—OH) groups attached to a benzene ring. A gallol moiety can comprise the structure:

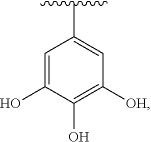 [SMILES: *c1cc(O)c(O)c(O)c1]

wherein the

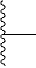

is the point of attachment to the compound.

A catechol- or gallol-containing compound can facilitate crosslinking with the macromolecule of the adhesive when cured, thus promoting strong adhesive properties. The chemistry of catechols and gallols is flexible in that, when formulated in compositions as described herein, these moieties are available (via oxidation or otherwise) to effectuate different bonds and/or interactions with a variety of substrates and/or compounds and facilitate crosslinking, including, for example, through the formation of hydrophobic interactions, hydrogen bonds, pi-pi interactions, salt bridges, and the like. Accordingly, the catechol and/or gallol moieties can readily react and/or cross-link with moieties of the macromolecule to form the adhesives hereof.

In certain embodiments, the one or more catechol-containing compound is any compound comprising one or more catechol moieties, preferably wherein the compound is biocompatible. In certain embodiments, the one or more catechol- or gallol-containing compounds can be catechol, vanillin, caffeic acid, juglone (5-hydroxy-1,4-naphthoquinone), catecholamine, resveratrol, 3,4-dihyroxyphenylacetic acid (DOPAC), catechin hydrate, 3,4-dihydroxy benzoic acid, 3,4-dihydroxybenzaldehyde, or any combination thereof.

In certain embodiments, the one or more gallol-containing compound is any compound comprising one or more gallol moieties, preferably wherein the compound is biocompatible. As used herein, “biocompatible” describes something that can be substantially non-toxic in the in vivo environment of its intended use and is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause a significantly adverse reaction or response. In certain embodiments, the one or more gallol-containing compounds comprises tannic acid (TA), gallic acid, gallotannins, ellagic acid, eugallol, 1,2,3-trihydroxybenzene, quercetin, gallol, or any combination thereof.

TA is a naturally occurring polyphenol and one of the cheapest sources of mussel-inspired chemistry). TA can have the structure:

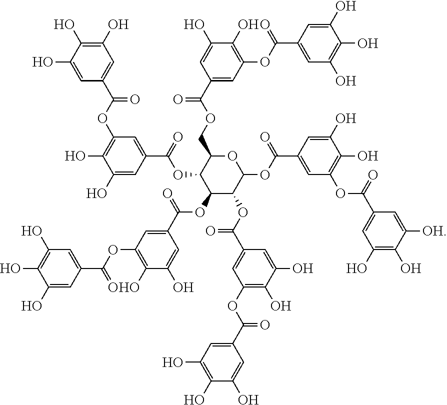 [SMILES: O=C(OC[C@H]1OC(OC(=O)c2cc(O)c(O)c(OC(=O)c3cc(O)c(O)c(O)c3)c2)[C@H](OC(=O)c2cc(O)c(O)c(OC(=O)c3cc(O)c(O)c(O)c3)c2)[C@@H](OC(=O)c2cc(O)c(O)c(OC(=O)c3cc(O)c(O)c(O)c3)c2)[C@@H]1OC(=O)c1cc(O)c(O)c(OC(=O)c2cc(O)c(O)c(O)c2)c1)c1cc(O)c(O)c(OC(=O)c2cc(O)c(O)c(O)c2)c1]

While TA is used herein in several of the Examples to provide those of ordinary skill in the art with a complete disclosure and description of the adhesives described herein, it will be appreciated that the adhesives hereof are not limited to TA and other catechol- and/or gallol-containing compounds can be employed to make and use the embodiments hereof.

The rings of the catechol and/or gallol moieties can be unsubstituted (except for the hydroxyl groups making up the functionality of the moieties and the attachment of the ring to the rest of the compound) or can be optionally substituted with additional functional groups. In certain embodiments, a ring of a gallol- or catechol- or gallol-containing compound is substituted with an electron withdrawing group, such as a nitro group (—NO₂).

The adhesive can be formulated at or around a neutral pH (for example, about pH=7). The adhesive can be formulated at or around a pH of at or about a pH of 6-8 (such as 6-8).

The term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 10% of the stated number or numerical range (e.g., +/−5% to 10% of the recited value) provided that one of ordinary skill in the art would consider equivalent to the recited value (e.g., having the same function or result). The term “substantially” can allow for a degree of variability in a value or range, for example, within 90%, within 95%, 99%, 99.5%, 99.9%, 99.99%, or 99.999% or more of a stated value or of a stated limit of a range, inclusive of the specified end points.

In certain embodiments, the adhesive comprises a pH of 5.8, a pH of 5.9, a pH of 6.0, a pH of 6.1, a pH of 6.2., a pH of 6.3, a pH of 6.4, a pH of 6.5, a pH of 6.6, a pH of 6.7, a pH of 6.8, a pH of 6.9, a pH of 7.0, a pH of 7.1, a pH of 7.2, a pH of 7.3, a pH of 7.4, a pH of 7.5, a pH of 7.6, a pH of 7.7, a pH of 7.8, a pH of 7.9, a pH of 8.0, a pH of 8.1, or a pH of 8.2. In certain embodiments, the macromolecule and/or the one or more catechol- or gallol-containing or gallol-containing compound can have a basic pH (e.g., as high as at or about a pH of 9.0), but when combined with TA (or another acidic catechol- or gallol-containing or gallol-containing compound), the prepared adhesive comprises a pH of at or about 6, at or about 7, or at or about 8.0.

In certain embodiments, there is more macromolecule (e.g., zein) in the adhesive (e.g., by weight of the prepared adhesive prior to curing) as compared to the one or more catechol- or gallol-containing compound(s). For example, the adhesive can comprise at or about 25-45 wt. % macromolecule (e.g., zein). In certain embodiments, the adhesive comprises at or about 25-45 wt. % macromolecule (e.g., zein) and at or about 5-30 wt. % catechol- or gallol-containing compound (e.g., TA) (of the uncured adhesive). In certain embodiments, the adhesive can comprise at or about 30-40 wt. % macromolecule (e.g., zein) and at or about 7-10 wt. % catechol- or gallol-containing compound (e.g., TA) (of the uncured adhesive). In certain embodiments, the adhesive comprises at or about 40 wt. % macromolecule and at or about 7 wt. % catechol- or gallol-containing compound (e.g., zein is 40% of the weight of the prepared adhesive prior to curing and 7% of the weight of the prepared adhesive is TA prior to curing). In certain embodiments, the adhesive can comprise at or about 24-35 wt. % macromolecule (e.g., zein) and at or about 18-22 wt. % catechol- or gallol-containing compound (e.g., TA) (of the uncured adhesive). In certain embodiments, the adhesive comprises at or about 26 wt. % macromolecule and at or about 21 wt. % catechol- or gallol-containing compound (of the uncured adhesive). In certain embodiments, the adhesive can comprise at or about 25-30 wt. % macromolecule (e.g., zein) and at or about 18-22 wt. % catechol- or gallol-containing compound (e.g., TA) (of the uncured adhesive). In certain embodiments, the adhesive comprises at or about 30 wt. % macromolecule and at or about 21 wt. % catechol- or gallol-containing compound (of the uncured adhesive).

Alternatively, there can be more catechol- or gallol-containing compound in the adhesive as compared to the macromolecule (e.g., by weight of the prepared adhesive prior to curing). In certain embodiments, the adhesive can comprise at or about 20-30 wt. % macromolecule (e.g., zein) and at or about 28-38 wt. % catechol- or gallol-containing compound (e.g., TA) (of the uncured adhesive). In certain embodiments, the adhesive comprises at or about 26 wt. % macromolecule and at or about 28 wt. % catechol- or gallol-containing compound (of the uncured adhesive).

In certain embodiments, the adhesive comprises a macromolecule, at least one catechol- or gallol-containing compound, and a solvent. As used herein, the macromolecule and at least one catechol- or gallol-containing compound taken together are referred to as the “solid components.”

The solvent can be an aqueous and/or organic solvent. The solvent can comprise water. The solvent can comprise an alcohol. The solvent can comprise an alcohol and water.

The alcohol of the solvent, when used, can comprise ethanol, isopropyl alcohol, or any other alcohol that may be appropriate for the desired use-case of the adhesive. In certain embodiments, the solvent is ethanol.

The components of the adhesive can be combined in appropriate ratios to yield strong tissue sealants and/or adhesives. In certain embodiments where the solvent comprises at least alcohol and water, the alcohol and water are present in at or about a (2-5):1 ratio by volume relative to each other. In certain embodiments, the alcohol and water are present in at or about a 5:1 ratio by volume. In certain embodiments, the alcohol and water are present in at or about a (3-5):1 ratio by volume. In certain embodiments, the alcohol and water are present in at or about a (4-5):1 ratio by volume. In certain embodiments, the alcohol and water are present in at or about a 4:1 ratio by volume. In certain embodiments, the alcohol and water are present in at or about a (3-4):1 ratio by volume. In certain embodiments, the alcohol and water are present in at or about a (2-4):1 ratio by volume. In certain embodiments, alcohol and water are present in at or about a 3:1 ratio by volume. In certain embodiments, the alcohol and water are present in at or about a (2-3):1 ratio by volume. In certain embodiments, the alcohol and water are present in at or about a 2:(1-2) ratio by volume. In certain embodiments, the alcohol and water are present in at or about a 2:1 ratio by volume. In certain embodiments, the alcohol and water ratio are present in at or about a 1:1 ratio by volume. In certain embodiments, the alcohol and water ratio are present in at or about 1:(2-1) ratio by volume. The ratio ranges specified herein are inclusive of the stated end points.

Further, the ratio of solid components to solvent can be at or about (45%-80%):(55%-20%) by weight of the total adhesive. This ratio can be modified as desired to the extent the adhesive remains capable of establishing a bond and/or crosslinking with a substrate when applied thereto.

In certain embodiments, the solid components account for about 45% of the weight of the adhesive, and the solvent accounts for about 55% of the weight of the adhesive (such as solid components to solvent percent weight ratio of 45:55). In certain embodiments, the solid component to solvent percent weight ratio is at or about 50:50. In certain embodiments, the solid component to solvent percent weight ratio is at or about 55:45. In certain embodiments, the solid component to solvent percent weight ratio is at or about 60:40. In certain embodiments, the solid component to solvent percent weight ratio is at or about 65:35. In certain embodiments, the solid component to solvent percent weight ratio is at or about 70:30. In certain embodiments, the solid component to solvent percent weight ratio is at or about 75:25. In certain embodiments, the solid component to solvent percent weight ratio is at or about 80:20. The ratio ranges specified herein are inclusive of the stated end points.

In certain embodiments, the weight percentage of the solid components is about 45-80 w-t. % of the prepared adhesive prior to curing. In certain embodiments, the weight percentage of the solid components is about 50-75 wt. % of the prepared adhesive prior to curing. In certain embodiments, the weight percentage of the solid components is about 55-70 wt. % of the prepared adhesive prior to curing. In certain embodiments, the weight percentage of the solid components is about 60-65 wt. % of the prepared adhesive prior to curing. In certain embodiments, the weight percentage of the solid components is about 45-80 wt. % of the prepared adhesive prior to curing (inclusive of the end points of the specified range).

In certain embodiments, the solid component to solvent wt. % ratio is such that substantially all of the solid components are dissolved (and remain dissolved at rest) in the solvent. In certain embodiments, the solid component to solvent percent weight ratio is such that the majority of the solid components are dissolved in the solvent, albeit some solid components can remain suspended therein. It will be appreciated that the higher the wt. % of solid components of the prepared adhesive prior to curing, the more viscous the solution will be. Accordingly, the wt. % ratio of solid components to solvent can be modified to achieve a particular viscosity as may be beneficial for a particular use case of the adhesive.

In certain embodiments, the combined weight percentage of the total solid components and solvent constitutes at least about 90% (e.g., 90%) of the total weight of the adhesive composition prior to curing. In certain embodiments, the combined weight percentage of the total solid components and solvent constitutes at least about 91% (e.g., 91%) of the total weight of the adhesive composition prior to curing. In certain embodiments, the combined weight percentage of the total solid components and solvent constitutes at least about 92% (e.g., 92%) of the total weight of the adhesive composition prior to curing. In certain embodiments, the combined weight percentage of the total solid components and solvent constitutes at least about 93% (e.g., 93%) of the total weight of the adhesive composition prior to curing. In certain embodiments, the combined weight percentage of the total solid components and solvent constitutes at least about 94% (e.g., 94%) of the total weight of the adhesive composition prior to curing. In certain embodiments, the combined weight percentage of the total solid components and solvent constitutes at least about 95% (e.g., 95%) of the total weight of the adhesive composition prior to curing. In certain embodiments, the combined weight percentage of the total solid components and solvent constitutes at least about 96% (e.g., 96%) of the total weight of the adhesive composition prior to curing. In certain embodiments, the combined weight percentage of the total solid components and solvent constitutes at least about 97% (e.g., 97%) of the total weight of the adhesive composition prior to curing. In certain embodiments, the combined weight percentage of the total solid components and solvent constitutes at least about 98% (e.g., 98%) of the total weight of the adhesive composition prior to curing. In certain embodiments, the combined weight percentage of the total solid components and solvent constitutes at least about 99% (e.g., 99%) of the total weight of the adhesive composition prior to curing. In certain embodiments, the combined weight percentage of the total solid components and solvent constitutes about 100% (e.g., 100%) of the total weight of the adhesive composition prior to curing.

In certain embodiments, the adhesive comprises zein, TA, and a solvent (e.g., ethanol and water). In certain embodiments, the adhesive consists of only four ingredients, namely, a macromolecule, a catechol- or gallol-containing compound, ethanol, and water, and does not require any additional curing agent or preservative. In certain embodiments, the adhesive consists of only four ingredients, namely, zein, TA, ethanol, and water, and does not require any additional curing agent or preservative. In certain embodiments, the adhesive can further comprise one or more fillers. Non-limiting examples of such fillers include hydroxyapatite, calcium carbonate, calcium phosphate, magnesium phosphate, laponite, montmorillonite, or a combination thereof.

The adhesives hereof can be tuned for specific applications (e.g., for medical and/or surgical applications, for example, to a particular tissue when used as a tissue sealant). Varying the overall solvent concentration, ratio of the two solvents (e.g., ethanol and water), the ratio of macromolecule to the one or more catechol- or gallol-containing compounds, and varying the pH of the resulting adhesive can affect the properties of the resulting adhesive. For example, and without limitation, where a strong bond is desired, the adhesive can be formulated with a higher wt. % of solid components as compared to solvent wt. %. Alternatively, where a thinner adhesive is desired, the adhesive can be formulated with a lower wt. % of solid components as compared to solvent wt. %.

In certain embodiments, the adhesive can further comprise an iron crosslinker, an interfacial crosslinker, or both an iron crosslinker and an interfacial crosslinker.

The iron crosslinker can comprise a chemical compound that contains an iron atom and can be useful to facilitate the formation of crosslinks and/or bridges between the macromolecule and one or more catechol- or gallol-containing compound of the adhesive, and/or the underlying substrate to which the adhesive is applied. In certain embodiments, the iron crosslinker comprises an iron in a 3+ oxidation state. In certain embodiments, the iron crosslinker comprises ferrous sulfate (FeSO₄), ferric chloride (FeCl₃), an iron oxide nanoparticle, ferric nitrate (Fe(NO₃)₃), iron(III) acetylacetonate (Fe(acac)₃ or Fe(C₅H₇O₂)₃), potassium ferrate (K₂FeO₄), or any combination thereof. The interfacial crosslinker can comprise a non-toxic enzyme or other molecule that can catalyze the formation of covalent bonds between proteins, leading to crosslinking. The interfacial crosslinker can comprise transglutaminase (TGnase) (including without limitation microbial TGnase (mTG)), ethyl-3-(3-dimethylaminopropyl)carbodiimide hydrochloride (EDC), N-hydroxy succinimide (NHS), an elastin-like polypeptide (ELP), polyethylene glycol (PEG), a dendrimer, glutaraldehyde, and/or photocrosslinkers. In certain embodiments, the interfacial crosslinker comprises TA.

TGnase is an enzyme that plays a role in various biological processes, and particularly in the formation of covalent bonds between proteins. For example, in nature, TGnase primarily catalyze the formation of an isopeptide bond between γ-carboxamide groups of glutamine residue side chains and the ε-amino groups of lysine residue side chains with subsequent release of ammonia.

TGnase is found in various organisms including humans and microbes. For example, the TGnase can comprise a plant, recombinant animal, or microbe derived TGnase. In certain embodiments, the TGnase can be extracted from one or more of Streptoverticillium Baldaccii, a Streptomyces Hygroscopicus strain, or Escherichia Coli. In certain embodiments, the TGnase is any type of calcium dependent or independent TGnase. In certain embodiments the interfacial crosslinker comprises one or more synthesized polymer sequences featuring a TGnase cross-linking site.

In certain embodiments, the TGnase has a pH in a range from about 5 to about 8. The pH range specified is inclusive of the end points. The TGnase can be a pharmaceutically acceptable salt of TGnase, a hydrate, N-oxide, and/or solvate thereof. The term “solvate” means a protein, or a salt thereof, that further includes a stoichiometric or non-stoichiometric amount of solvent bound by non-covalent intermolecular forces. Where the solvent is water, the solvate is a hydrate. It will be appreciated that certain functional groups, such as a hydroxy, amino, and like groups, can form complexes and/or coordination compounds with water and/or various solvents, in the various physical forms of TGnase.

The TGnase can be commercial food-grade TGnase, which can be advantageous where, for example, the adhesive is used as a medical sealant. When the adhesive is used as a medical/surgical sealant, the TGnase can catalyze a reaction between glutamine residues in zein and lysine residues on the targeted tissue (e.g., damaged or compromised tissue). Thus, TGnase can act as an interfacial crosslinker that binds to the macromolecule of the adhesive as well as the damaged tissue itself, thus reinforcing the adhesion strengths. This combination of interactions from the macromolecule (e.g., zein), the catechol- or gallol-containing compound (e.g., TA), and TGnase with the tissue can enhance the overall adhesion strength of sealants in use. Other known iron crosslinkers and/or interfacial crosslinkers can also be used with the adhesive to achieve the same or similar effect as described herein.

The interfacial crosslinker can optionally be in a composition comprising at least one other substance, such as a stabilizer or filler for example. Non-limiting examples of such materials include maltodextrin, hydrolyzed skim milk protein or any other protein substance, sodium chloride, trisodium phosphate, sodium caseinate, or lactose, hydroxyapatite, calcium carbonate, calcium phosphate, magnesium phosphate, laponite, montmorillonite, or a combination thereof.

In some embodiments, the adhesive can further comprise an oxidant. The oxidant can comprise sodium periodate, tetrabutylammonium periodate, potassium permanganate, di-tert-butyl peroxide, hydrogen peroxide, cumene hydroperoxide, 2-butanone hydroperoxide, chromate, dichromate, or any combination thereof.

In certain embodiments, the adhesives are as strong as commercial cyanoacrylate (Super Glue) when measured under similar conditions on aluminum substrates. In certain embodiments, the adhesive (e.g., when cured) has an adhesive strength/value of about 1.5-2.0 MPa, about 2.0-3.0 MPa, 3.0-4.0 MPa, about 4.0-5.0 MPa, about 5.0-6.0 MPa, about 6.0-7.0 MPa, about 6.5-7.0 MPa, about 7.0-7.5 MPa, about 7.5-8.0 MPa, about 8.0-8.5 MPa, about 8.5-9.0 MPa, about 9.0-9.5 MPa, about 9.5-10.0 MPa, about 10-15 MPa, about 15-20 MPa, about 20-21 MPa, or greater than about 21 MPa. In certain embodiments, the adhesive has a tensile strength of up to about 20 MPa (such as 20 MPa). In certain embodiments, the adhesive has an adhesion value of about 1.5 MPa. In certain embodiments, the adhesive has an adhesion value of about 3.0 MPa. The adhesives can also exhibit a good degree of water resistance.

Tissue sealants are also provided that comprise a biologically acceptable, optionally rapidly gelling (e.g., where the adhesive comprises an iron crosslinker and/or interfacial crosslinker, and/or oxidant), formulation having a specified range of adhesive and cohesive properties. The tissue sealant hereof typically gels and/or bonds rapidly upon contact with tissue, such as, for example, tissue containing collagen or other proteins. The tissue sealant can comprise zein, TA, and a solvent. In certain embodiments, the solvent comprises ethanol and water. The tissue sealant can comprise any adhesive described herein. The tissue sealant can consist of zein, tannic acid, and a solvent (and no other substantial components). The tissue sealant can further comprise TGnase or another crosslinking agent. The tissue sealant can further comprise an iron crosslinker and/or an interfacial crosslinker. The tissue sealant can further comprise an oxidant. The tissue sealant can further comprise a filler.

The tissue sealant can be suitable for treatment of tissue damage. The tissue can be any tissue type including, without limitation, skin, heart tissue, dura mater, stomach tissue, lung tissue, liver tissue, bone and/or cartilage, intestines, blood vessel tissue, aortic tissue, lymph tissue, muscle tissue, connective tissue, and the like. The tissue sealant can be suitable for treatment of soft tissue lesion. The tissue sealant can be suitable for treatment of injured or traumatized cartilage, bone, and/or cartilage-bone defects. The tissue sealant can be suitable for treatment of any type of biological tissue. In certain embodiments, the tissue sealant can be sprayed onto a region of a lymph node or transected lymph duct. The tissue sealant can be suitable for the treatment of nerve tissue.

In certain embodiments, the tissue sealant can be useful for applications such as sealing tissue damage (e.g., a wound or laceration), reinforcing surgical sutures and/or surgical staples, adhering and/or reinforcing a graft, and/or for any type of sealant and/or adherent activity.

When applied to a variety of tissue substrates (e.g., organs), the adhesives and tissue sealants can withstand pressures higher than normal physiological pressure experienced in most of the organs tested (see Examples below). The adhesives and tissue sealants hereof also performed better than the commercial gold standard fibrin tissue sealant (Tisseel) when tested on most tissues.

When oxidized, the one or more catechol- or gallol-containing compounds of the adhesives/tissue sealants hereof can react with functional groups in the macromolecule (e.g., zein protein), thus forming the cross-linked tissue sealant. Moreover, un-crosslinked catechol- or gallol-containing compounds (e.g., TA) can further interact with functional groups on the tissue itself. The functional groups originating from the amino acid residues in proteins, polypeptides, and fatty acids on tissues are the primary source of covalent interactions between tissues and adhesives/sealants. Basic amino acids like lysine, histidine, and polypeptides present in tissue provide amino groups, and acidic amino acids like glutamic acid and fatty acids present in tissue provide carboxylic groups to bond with relevant functional groups of the sealants (e.g., the hydroxyl groups of the catechol- or gallol-containing compounds of the adhesive). Moreover, other functional groups such as imidazole from histidine and thiols from cysteine are also available to bond with relevant functional groups on the sealants. Amines are by far the most widely targeted functional groups owing to their higher abundance and reactivity. Other functional groups on grafts and tissues can also be crosslinked with the adhesive, including without limitation sulfonate groups and phosphates (which can interact with catechol and gallol moieties through coordination bonds) and hydroxyl groups (which can interact with catechol and gallol moieties to form hydrogen bonds).

The gallol groups in TA, for example, can oxidize to quinones, which can react with amines on targeted tissues (e.g., damaged tissues) via various pathways. Benzene rings of quinones can undergo Michael addition reaction with amines, thiols, or imidazole on tissues. Quinones can form Schiff base linkages between carbonyl groups and amines. The majority of these interactions are based on Michael type addition. Moreover, physical crosslinking via hydrogen bonding also occurs and can also be a significant factor contributing towards at least short-term adhesion.

A method of sealing a damaged tissue of a subject is also provided. In certain embodiments, the method comprises applying an adhesive or tissue sealant hereof to a damaged tissue (e.g., a targeted site of a damaged tissue). The damaged tissue can be any tissue to which the adhesive or tissue sealant can be suitably applied. The damaged tissue can be mammalian tissue. The damaged tissue can be in vivo (e.g., as the adhesive and/or tissue sealant can be biodegradable, it is suitable for such applications). The damaged tissue can be any type of in vivo mammalian tissue. The damaged tissue can be in an oral environment (e.g., in a mouth of the subject).

The damaged tissue can be selected from the group consisting of sausage casing/intestine, skin, heart tissue, stomach tissue, lung tissue, nerve tissue, liver tissue, dura mater, and an aorta. The damaged tissue can have any damage including, for example, a puncture, a wound, an abrasion, a cut, or any other tissue damage where application of a sealant/adhesive can be beneficial. The adhesive/sealant can be used to help prevent, for example, leaks (e.g., where the damaged tissue is under pressure), reduce blood loss, and/or be used in conjunction with or in place of mechanical methods such as sutures or staples.

In certain embodiments, the method can further comprise applying an iron crosslinker and/or an interfacial crosslinker to the damaged tissue to form a first layer and applying the adhesive to the first layer. The iron crosslinker can comprise a chemical compound that contains an iron atom and can be useful to facilitate the formation of crosslinks and/or bridges between the macromolecule and one or more catechol- or gallol-containing compound of the adhesive or tissue sealant, and/or the underlying tissue to which the adhesive is applied. In certain embodiments, the iron crosslinker comprises an iron in a 3+ oxidation state. In certain embodiments, the iron crosslinker comprises ferrous sulfate (FeSO₄), ferric chloride (FeCl₃), an iron oxide nanoparticle, ferric nitrate (Fe(NO₃)₃), iron(III) acetylacetonate (Fe(acac)₃ or Fe(C₅H₇O₂)₃), potassium ferrate (K₂FeO₄), or any combination thereof.

The interfacial crosslinker can comprise a non-toxic enzyme or other molecule that can catalyze the formation of covalent bonds between proteins, leading to crosslinking. The interfacial crosslinker can comprise TGnase (including without limitation mTG), an ELP, PEG, a dendrimer, glutaraldehyde, and/or photocrosslinkers. In certain embodiments, the interfacial crosslinker comprises TA.

The interfacial crosslinker can be a powder. The interfacial crosslinker can be a viscous solution. The viscous solution of the interfacial crosslinker can, for example, comprise the interfacial crosslinker and water.

A “subject” can be a mammal. For example, and without limitation, the subject can be a human patient, a laboratory animal, such as a rodent (e.g., mouse, rat, or hamster), a rabbit, a monkey, a chimpanzee, a domestic animal, such as a dog, a cat, or a rabbit, an agricultural animal, such as a cow, a horse, a pig, a sheep, or a goat, or a wild animal in captivity, such as a bear, a panda, a lion, a tiger, a leopard, an elephant, a zebra, a giraffe, a gorilla, a dolphin, or a whale. The terms “subject” and “patient” are used interchangeably herein.

In certain embodiments of the method, when the adhesive or tissue sealant is applied to a damaged tissue, the one or more catechol- or gallol-containing compounds interact with functional groups of the damaged tissue to further facilitate and/or support an adhesive bond with the damaged tissue.

The method can further comprise applying one or more sutures to the damaged tissue. The method can further comprise applying a graft to the damaged tissue. The graft can be a synthetic material and/or a biological material as is known in the art. In certain embodiments, the graft comprises separately grown tissue or processed tissue patches, growth scaffolds, or the like. The graft can be a porous graft (e.g., a mesh graft such as a hernia mesh), a thin-walled graft, a xenograft, a full-thickness graft, or any other graft for replacement of damaged or diseased tissue, to enhance function, and/or improve aesthetics.

The adhesive or tissue sealant can be applied to the damaged tissue before or after the suture(s) and/or graft, as desired. In certain embodiments, the adhesive or tissue sealant is applied to a graft, and the graft comprising the adhesive or tissue sealant thereon is then applied to the damaged tissue. In certain embodiments, the adhesive or tissue sealant is applied to the damaged tissue before the graft is applied to the damaged tissue (e.g., the graft can be applied to the damaged tissue that already has the adhesive or sealant applied thereto). In certain embodiments, a graft is applied to the damaged tissue and, thereafter, a layer of adhesive or tissue sealant is applied over the non-tissue facing surface of the graft and onto tissue surrounding the graft. In such embodiments, the adhesive or tissue sealant need not be applied directly to the damaged tissue site, but rather can be used to adhere (and/or reinforce) the graft's placement over the damage tissue.

The method can further comprise curing the adhesive or tissue sealant and/or allowing the adhesive or tissue sealant to cure. The adhesive or tissue sealant will cure upon removal of the solvent, which leads to the oxidation of the hydroxyl functional groups of the catechol- or gallol-containing compounds thereof and, thus, crosslinking of the adhesive components with each other and/or the underlying tissue and/or graft. The solvent can be removed from the adhesive or tissue sealant by exposing the adhesive to oxygen. The solvent can be removed from the adhesive or tissue sealant by exposing the adhesive to heat. In certain embodiments, curing the adhesive or tissue sealant comprises removing or facilitating removal of the solvent from the adhesive or tissue sealant. Curing the adhesive can comprise exposing the adhesive or tissue sealant to oxygen and/or heat.

Application of heat to the adhesive or tissue sealant in conjunction with or in lieu of oxygen exposure can reduce the time required for the adhesive to cure as it can facilitate evaporation or removal of the solvent therein. In certain embodiments, applying heat to cure the adhesive or tissue sealant comprises exposing the adhesive or tissue sealant to a temperature of between about 31.0° C. to about 42.0° C. (e.g., incubating the adhesive or tissue sealant at or between 31.0° C. to 42.0° C.). This temperature range roughly aligns with body temperature and/or skin temperature (i.e. 87.8° F. to 104° F.) and, as such, is useful for where the adhesive is applied in vivo. In certain embodiments, curing the adhesive comprises exposing comprises exposing the adhesive or tissue sealant to a temperature of about 32.0° C. (such as 32.0° C.). In certain embodiments, curing the adhesive comprises exposing comprises exposing the adhesive or tissue sealant to a temperature of about 33.0° C. (such as 33.0° C.). In certain embodiments, curing the adhesive comprises exposing comprises exposing the adhesive or tissue sealant to a temperature of about 34.0° C. (such as 34.0° C.). In certain embodiments, curing the adhesive comprises exposing comprises exposing the adhesive or tissue sealant to a temperature of about 35.0° C. (such as 35.0° C.). In certain embodiments, curing the adhesive comprises exposing comprises exposing the adhesive or tissue sealant to a temperature of about 36.0° C. (such as 36.0° C.). In certain embodiments, curing the adhesive comprises exposing comprises exposing the adhesive or tissue sealant to a temperature of about 37.0° C. (such as 37.0° C.). In certain embodiments, curing the adhesive comprises exposing comprises exposing the adhesive or tissue sealant to a temperature of about 38.0° C. (such as 38.0° C.). In certain embodiments, curing the adhesive comprises exposing comprises exposing the adhesive or tissue sealant to a temperature of about 39.0° C. (such as 39.0° C.). In certain embodiments, curing the adhesive comprises exposing comprises exposing the adhesive or tissue sealant to a temperature of about 40.0° C. (such as 40.0° C.). In certain embodiments, curing the adhesive comprises exposing comprises exposing the adhesive or tissue sealant to a temperature of about 41.0° C. (such as 41.0° C.). In certain embodiments, curing the adhesive comprises exposing comprises exposing the adhesive or tissue sealant to a temperature of about 42.0° C. (such as 42.0° C.). The temperature ranges described herein are inclusive of the stated end points thereof.

In certain embodiments where the adhesive comprises a photocrosslinker or photoinitiator filler, the adhesive can be cured by application of light thereto, for example using methods commonly known in the art.

The adhesive and/or tissue sealant hereof can cure in an in vivo environment, for example, when subjected to one or more biofluids. In certain embodiments, the adhesive or tissue sealant is allowed to cure in the presence of a biofluid. The biofluid can be any mammalian biofluid. The biofluid can be gastric fluid. The biofluid can be blood, plasma, or serum. The biofluid can be bile. The biofluid can be tears. The biofluid can be interstitial fluid. The biofluid can be sweat. The term “biofluid” can also include mixtures of biofluids such as sweat and blood, or bile and interstitial fluid. In certain embodiments, for example, where the damaged tissue is in an oral environment, the method comprises allowing the adhesive or tissue sealant to cure in saliva.

In certain embodiments where the adhesive or tissue sealant is applied to a graft prior to application to a damaged tissue, the method can further comprise allowing the adhesive or tissue sealant applied to a graft to cure (at least partially) before applying the adhesive/tissue sealant and graft to the damaged tissue. In certain embodiments, the adhesive/tissue sealant is allowed to cure (e.g., is exposed to oxygen, heat, and/or light) for less than about 15 minutes (such as less than 15 minutes) before applying the adhesive or tissue sealant and graft to the damaged tissue. In certain embodiments, the adhesive/tissue sealant is allowed to cure for less than about 40 minutes (such as less than 40 minute) before applying the adhesive or tissue sealant and graft to the damaged tissue.

The adhesive and/or tissue sealant can cure in less than 15 minutes. The adhesive and/or tissue sealant can cure in less than 13 minutes, less than 12 minutes, less than 11 minutes, less than 10 minutes, less than 9 minutes, less than 8 minutes, less than 7 minutes, less than 6 minutes, less than 5 minutes, less than 4 minutes, less than 3 minutes, less than 2 minutes, and/or less than 1 minute.

Any volume of adhesive or tissue sealant can be used as may be appropriate for a particular application and/or as desired. In certain embodiments, an amount of adhesive or tissue sealant applied to the damaged tissue is about 10 μl to 25 ml in volume (such as 10 μl to about 25 ml, about 10 μl to 25 ml, or between 10 μl-25 ml). In certain embodiments, the amount of adhesive or tissue sealant applied to the damaged tissue is at or about 10 μl such as, for example, when used for small nerve repair. In certain embodiments, the amount of adhesive or tissue sealant applied to the damaged tissue is at or about 25 ml such as, for example, when used for large tissue patch repairs. In certain embodiments, the amount of adhesive or tissue sealant applied to the damaged tissue is at or about 80 μl to 150 μl in volume. In certain embodiments, the amount of adhesive or tissue sealant applied to the damaged tissue is at or about 150 μl in volume.

Methods for formulating an adhesive/tissue sealant are also provided. Such a method can comprise forming a first solution by combining a first amount of zein, a second amount of one or more catechol- or gallol-containing compounds (e.g., TA, etc.), and a third amount of a solvent; adjusting the pH of the first solution to between about 6-8 (e.g., such as 6-8) if needed; and curing the first solution to substantially remove the solvent therefrom. Curing the first solution can comprise incubating the first solution at a temperature of between about 31.0° C. to 42.0° C. (e.g., 31.0, 32.0° C., 33.0° C., 34.0° C., 35.0° C., 36.0° C., 37.0° C., 38.0° C., 39.0° C., 40.0° C., 41.0° C., or 42.0° C.). In certain embodiments, curing the first solution comprises exposing the first solution to oxygen. In certain embodiments, curing the first solution comprises light curing. Light curing or curing with light generally refers to applying a light to the adhesive or tissue sealant for providing light curing wavelengths configured to cure the adhesive or tissue sealant (e.g., using light emitting diodes (LEDs), quartz-tungsten-halogen (QTH) bulbs, or another activating light source). Where the first solution comprises a photocroslinker or photoinitiator filler, for example, such filler can be photosensitive and, when irradiated by light, can facilitate crosslinking of the components of the first solution and/or the damaged tissue.

The pH of the first solution can be adjusted to at or about 6.0, at or about 6.5, at or about 7.0, at or about 7.5, or at or about 8.0, if needed.

The solvent can comprise water. The solvent can comprise an alcohol. The solvent can comprise an alcohol and water. The alcohol of the solvent, when used, can comprise ethanol, isopropyl alcohol, or any other alcohol that may be appropriate for the desired use-case of the adhesive. In certain embodiments, the solvent is ethanol. In certain embodiments, the ethanol to water ratio of the first solution is at or about 3:1 or any of the other ratios described herein.

In certain embodiments, the first solution can further comprise an iron crosslinker, an interfacial crosslinker, or both an iron crosslinker and an interfacial crosslinker. The interfacial crosslinker can be TGnase. In certain embodiments, the first solution can further comprise a filler. In certain embodiments, the first solution can further comprise an oxidant.

The one or more catechol- or gallol-containing compound can be any catechol- or gallol-containing compounds described herein. In certain embodiments, the catechol- or gallol-containing compound comprises catechol, vanillin, caffeic acid, juglone (5-hydroxy-1,4-naphthoquinone), catecholamine, resveratrol, DOPAC, catechin hydrate, 3,4-dihydroxy benzoic acid, 3,4-dihydroxybenzaldehyde, TA, gallic acid, gallotannins, ellagic acid, eugallol, 1,2,3-trihydroxybenzene, quercetin, gallol, or any combination thereof.

The method can further comprise applying the incubated first solution to a layer of a second solution comprising the interfacial crosslinker. For example, the second solution can comprise TGnase (e.g., as a powder or as a viscous solution comprising TGnase and a solvent such as water). The TGnase can be commercial food-grade TGnase. In certain embodiments, the second solution comprises a viscous solution of TGnase and a solvent (e.g., water). The TGnase can be any TGnase described herein.

The first solution can comprise any of the adhesives described herein. The first solution can comprise at or about 25-45 wt. % zein and at or about 5-30 wt. % catechol- or gallol-containing compound (e.g., TA). In certain embodiments, the first solution can comprise at or about 30-40 wt. % zein and at or about 7-10 wt. % catechol- or gallol-containing compound (e.g., TA). In certain embodiments, the first solution comprises at or about 40 wt. % zein and at or about 7 wt. % catechol- or gallol-containing compound. In certain embodiments, the first solution can comprise at or about 24-35 wt. % zein and at or about 18-22 wt. % catechol- or gallol-containing compound (e.g., TA). In certain embodiments, the first solution comprises at or about 26 wt. % zein and at or about 21 wt. % catechol- or gallol-containing compound. In certain embodiments, the first solution comprises at or about 25-30 wt. % zein and at or about 18-22 wt. % catechol- or gallol-containing compound (e.g., TA). In certain embodiments, the first solution comprises at or about 30 wt. % zein and at or about 21 wt. % catechol- or gallol-containing compound.

Alternatively, there can be more catechol- or gallol-containing compound in the first solution as compared to the zein (e.g., by weight of the prepared adhesive prior to curing). In certain embodiments, the first solution comprises at or about 20-30 wt. % zein and at or about 28-38 wt. % catechol- or gallol-containing compound (e.g., TA). In certain embodiments, the first solution comprises at or about 26 wt. % zein and at or about 28 wt. % catechol- or gallol-containing compound. The percentages of the components of the first solution can be any of the other percentages described herein.

The first solution can further comprise TGnase or another interfacial crosslinker, an iron crosslinker, or both an interfacial crosslinker and an iron crosslinker.

In certain embodiments where the solvent of the first solution comprises at least alcohol and water, the alcohol and water are present in at or about a (2-5):1 ratio by volume relative to each other. In certain embodiments, the alcohol and water are present in at or about a 5:1 ratio by volume. In certain embodiments, the alcohol and water are present in at or about a (3-5):1 ratio by volume. In certain embodiments, the alcohol and water are present in at or about a (4-5):1 ratio by volume. In certain embodiments, the alcohol and water are present in at or about a 4:1 ratio by volume. In certain embodiments, the alcohol and water are present in at or about a (3-4):1 ratio by volume. In certain embodiments, the alcohol and water are present in at or about a (2-4):1 ratio by volume. In certain embodiments, alcohol and water are present in at or about a 3:1 ratio by volume. In certain embodiments, the alcohol and water are present in at or about a (2-3):1 ratio by volume. In certain embodiments, the alcohol and water are present in at or about a 2:(1-2) ratio by volume. In certain embodiments, the alcohol and water are present in at or about a 2:1 ratio by volume. In certain embodiments, the alcohol and water ratio are present in at or about a 1:1 ratio by volume. In certain embodiments, the alcohol and water ratio are present in at or about 1:(2-1) ratio by volume. The ratio ranges specified herein are inclusive of the stated end points.

Further, the ratio of solid components to solvent of the first solution can be at or about (45%-80%):(55%-20%) by weight of the total first solution. This ratio can be modified as desired to the extent the resulting adhesive is capable of establishing a bond and/or crosslinking with a substrate when applied thereto.

In certain embodiments, the solid components of the first solution account for about 45% of the weight of the first solution, and the solvent accounts for about 55% of the weight of the first solution (such as solid components to solvent percent weight ratio of 45:55). In certain embodiments, the solid component to solvent percent weight ratio is at or about 50:50. In certain embodiments, the solid component to solvent percent weight ratio is at or about 55:45. In certain embodiments, the solid component to solvent percent weight ratio is at or about 60:40. In certain embodiments, the solid component to solvent percent weight ratio is at or about 65:35. In certain embodiments, the solid component to solvent percent weight ratio is at or about 70:30. In certain embodiments, the solid component to solvent percent weight ratio is at or about 75:25. In certain embodiments, the solid component to solvent percent weight ratio is at or about 80:20. The ratio ranges specified herein are inclusive of the stated end points.

In certain embodiments, the weight percentage of the solid components is about 45-80 wt. % of the prepared first solution prior to curing. In certain embodiments, the weight percentage of the solid components is about 50-75 wt. % of the prepared first solution prior to curing. In certain embodiments, the weight percentage of the solid components is about 55-70 wt. % of the prepared first solution prior to curing. In certain embodiments, the weight percentage of the solid components is about 60-65 wt. % of the prepared first solution prior to curing. In certain embodiments, the weight percentage of the solid components is about 45-80 wt. % of the prepared first solution prior to curing (inclusive of the end points of the specified range).

In certain embodiments, the solid component to solvent wt. % ratio is such that substantially all of the solid components are dissolved (and remain dissolved at rest) in the solvent. In certain embodiments, the solid component to solvent percent weight ratio is such that the majority of the solid components are dissolved in the solvent, albeit some solid components can remain suspended therein. It will be appreciated that the higher the wt. % of solid components of the prepared first solution prior to curing, the more viscous the solution will be. Accordingly, the wt. % ratio of solid components to solvent can be modified to achieve a particular viscosity of the first solution as may be beneficial for a particular application.

In certain embodiments, the combined weight percentage of the total solid components and solvent constitutes at least about 90% (e.g., 90%) of the total weight of the first solution prior to curing. In certain embodiments, the combined weight percentage of the total solid components and solvent constitutes at least about 91% (e.g., 91%) of the total weight of the first solution prior to curing. In certain embodiments, the combined weight percentage of the total solid components and solvent constitutes at least about 92% (e.g., 92%) of the total weight of the first solution prior to curing. In certain embodiments, the combined weight percentage of the total solid components and solvent constitutes at least about 93% (e.g., 93%) of the total weight of the first solution prior to curing. In certain embodiments, the combined weight percentage of the total solid components and solvent constitutes at least about 94% (e.g., 94%) of the total weight of the first solution prior to curing. In certain embodiments, the combined weight percentage of the total solid components and solvent constitutes at least about 95% (e.g., 95%) of the total weight of the first solution prior to curing. In certain embodiments, the combined weight percentage of the total solid components and solvent constitutes at least about 96% (e.g., 96%) of the total weight of the first solution prior to curing. In certain embodiments, the combined weight percentage of the total solid components and solvent constitutes at least about 97% (e.g., 97%) of the total weight of the first solution composition prior to curing. In certain embodiments, the combined weight percentage of the total solid components and solvent constitutes at least about 98% (e.g., 98%) of the total weight of the first solution prior to curing. In certain embodiments, the combined weight percentage of the total solid components and solvent constitutes at least about 99% (e.g., 99%) of the total weight of the first solution prior to curing. In certain embodiments, the combined weight percentage of the total solid components and solvent constitutes about 100% (e.g., 100%) of the total weight of the first solution prior to curing.

The resulting adhesive and tissue sealant has various characteristics (e.g., modulus, stiffness, etc.). In certain embodiments, the method can further comprise adjusting the amounts and/or ratios of the components of the first solution (e.g., the amounts of one or more of the zein, TA, ethanol, and water) to tune characteristic(s) of the adhesive to correspond with mechanical properties of a targeted tissue.

A kit for preparing the disclosed adhesives or tissue sealants is also provided. In some embodiments, the kit comprises at least one of the adhesives or tissue sealants described herein, and instructions for use. As used herein, “instructions for use” means a publication, a recording, a diagram, or any other medium of expression used to communicate the usefulness of the adhesive or tissue sealant for one of the purposes set forth herein. The instructional material of the kit can, for example, be affixed to a container which contains the present adhesive and/or tissue sealant and/or components thereof or be shipped together with a container which contains the adhesive and/or tissue sealant and/or components thereof. Alternatively, the instructional material can be shipped separately from the container or provided on an electronically accessible form on an internet website with the intention that the instructional material and the adhesive or tissue sealant be used cooperatively by the recipient.

In certain embodiments, the kit includes a macromolecule, one or more catechol- or gallol-containing compounds, and a solvent (e.g., of any of the adhesives or tissue sealants described herein), and instructions for use. In certain embodiments, the macromolecule and one or more catechol- or gallol-containing compounds are hydrated by the solvent for immediate use, such as in a syringe device or vial. There, the oxygen can be purged from the syringe device or vial after loading the adhesive or tissue sealant components (i.e., the first solution) therein.

In certain embodiments, the macromolecule and one or more catechol- or gallol-containing compounds are hydrated in the solvent for immediate use, such as in a dual syringe device to form a precursor liquid that rapidly gels upon exposure to oxygen and/or heat.

In certain embodiments, the kit comprises a powdered form of one or more catechol- or gallol-containing compounds, the macromolecule, or both the catechol- or gallol-containing compound(s) and the macromolecule. Different components in powdered form can be stored in a separate vial(s) or containers apart from the other components of the adhesive or tissue sealant. The solvent can be stored in a separate vial or container. The amounts of each of the components can be measured to achieve the desired ratios thereof and/or viscosity and/or properties of the adhesive or tissue sealant. In certain embodiments, the macromolecule is hydrated with the solvent such as in a vial or other container, and the catechol- or gallol-containing compound(s) are housed within a separate vial or container. In such embodiments, the pH of the macromolecule and solvent solution can be as high as 9, especially where the catechol- or gallol-containing compound(s) comprises TA, which, upon mixing with the macromolecule and solvent mixture, will act to bring the pH of the first solution down to a pH of at or about 6 to about 8. In certain embodiments, addition of the TA to the macromolecule and solvent mixture brings the pH of the first solution down to a pH of about 7 (i.e., a neutral pH).

The kit can have a shelf life of about 18 months. As used herein, “shelf life” is the length of time that the adhesive, tissue sealant, and/or components thereof can be stored without becoming unfit for use or sale and/or ineffective at exhibiting adhesive properties. In certain embodiments, the kit has a shelf life of about 17 months. In certain embodiments, the kit has a shelf life of about 16 months. In certain embodiments, the kit has a shelf life of about 15 months. In certain embodiments, the kit has a shelf life of about 14 months. In certain embodiments, the kit has a shelf life of about 13 months. In certain embodiments, the kit has a shelf life of about 12 months. In certain embodiments, the kit has a shelf life of about 11 months.

### General

All patents, patent application publications, journal articles, textbooks, and other publications mentioned in the specification are indicative of the level of skill of those in the art to which the disclosure pertains.

In the above description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein may be applied to compounds and/or composition components that have configurations other than as specifically described herein. Indeed, it is expressly contemplated that the components of the adhesives of the present disclosure may be tailored in furtherance of the desired application thereof.

Unless defined otherwise, all technical and scientific terms used herein have the same meaning as commonly understood to one of ordinary skill in the art. The terms and expressions employed are used as terms of description and not of limitation. In this regard, where certain terms are defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Certain Definitions,” are used in the “Detailed Description,” such use is solely for ease of reference and is not intended to limit any disclosure made in one section to that section only; rather, any disclosure made under one subheading is intended to constitute a disclosure under each and every other subheading.

Although any methods and materials similar to or equivalent to those described herein can be used in the practice or testing of the subject of the present application, the preferred methods and materials are described herein.

When ranges are used herein for physical properties, such as weight percentages, or chemical properties, such as chemical formulae, all combinations and sub-combinations of ranges and specific embodiments therein are intended to be included, and each range is inclusive of the end points.

It is recognized that various modifications are possible within the scope of the disclosure. Thus, although the present disclosure has been specifically disclosed in the context of preferred embodiments and optional features, those skilled in the art may resort to modifications and variations of the concepts disclosed herein. Such modifications and variations are considered to be within the scope of the disclosure as claimed herein.

In describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

### Certain Definitions

The disclosure may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms.

Likewise, the singular forms “a,” “an,” and “the” include plural references unless the context clearly dictates otherwise. Thus, for example, references to “the method” include one or more methods and/or steps of the type, which are described and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

### EXAMPLES

The following examples serve to illustrate the present disclosure. The examples are not intended to limit the scope of the claimed invention.

### Materials

Zein (Molecular weight:19-22 kDa), Tannic acid and Iron (III) chloride (FeCl₃·6H₂O) were purchased from Sigma-Aldrich (St. Louis, MO). Ethanol (190 proof) was purchased from Decon Laboratories Inc. (King of Prussia, PA). RM Transglutaminase (Moo Gloo™) was purchased from commercial sources. All other reagents used were purchased from Thermo Fischer Scientific (Waltham, MA) unless stated otherwise. All the porcine tissues and organs used in this study were procured locally or from Animal Technologies Inc. (Tyler, TX).

For studies with the optimized zein sealants, the porcine skin tissue was prepared and affixed to aluminum adherents similarly to as described. Specifically, 40 μl of the sealant was applied onto each of the tissues and they were bonded in lap shear configuration. The adherents were cured for 30 minutes and 2 hours at 37° C. in a humidifier as explained previously. For 30 minutes cure time, the adherents were cured in humidifier for 20 minutes and then allowed to equilibrate at room temperature for 10 minutes.

### Example 1

### Synthesis of Sea-Mussel Inspired Tissue Sealants

To determine the formulations with highest adhesive strengths, concentration of zein was kept constant at 40 wt. %, 30 wt. % and 26 wt. %. Tannic acid concentrations were varied from 1 wt. % to 28 wt. %. When iron crosslinker was added, the concentration of FeCl₃·6H₂O was varied from 0.05 wt. % to 1 wt. %. The ratio of ethanol to water was maintained at 3:1.

To prepare the sealants, pre-determined amounts of all the components were combined manually using a wooden spatula. The pH of the prepared sealant was adjusted to pH 7 using 10 M NaOH solution. The sealant was then incubated at 37° C. for 24 hours to remove all the bubbles before testing. The as prepared sealant is amber colored initially, but the color intensifies to dark brown after 24 hours depending on the amount of tannic acid or crosslinker used.

### Example 2

### Lap Shear Methodology

Lap shear test was performed as per a modified version of the American Society of Testing and Materials (ASTM) standard F2255-05 as previously described. Standard Test Method for Strength Properties of Tissue Adhesives in Lap-Shear by Tension Loading, ASTM International Website (publicly available; last accessed Aug. 16, 2023); Matos-Perez et al., Polymer Composition and Substrate Influences on the Adhesive Bonding of a Biomimetic, Cross-Linking Polymer, J Am Chemical Society 134(22): 9498-9505 (2012). Lap shear measurements are a test of bonding strength in which the sealant or adhesive formulation is applied to overlapping tabs of tissue, cured, and then the force to pull the tabs apart is measured. The test can reflect adhesive and cohesive bonding.

Porcine skin tissue obtained from the butcher was cut into approximately 1.8 cm by 1.2 cm dimensions. They were then affixed to aluminum adherents using cyanoacrylate glue. The glue was allowed to dry for 24 hours after which the samples were immersed in phosphate buffered saline (PBS) for 24 hours to hydrate the porcine tissue samples.

Before application of the sealants, the tissue was blotted dry with paper towels. For initial studies for optimization of ethanol to water ratio, 50 μl of the sealant was applied onto one of the tissue substrates and the other substrate was laid on top immediately. The adherents were cured for 2 hours at 37° C. in a humidifier; specifically, in a humidifier for 1.5 hours and allowed to equilibrate at room temperature for remaining 30 minutes. A lead weight of about 40 g was applied onto the adherents to apply pressure during curing.

### Example 3

### Burst Pressure Methodology

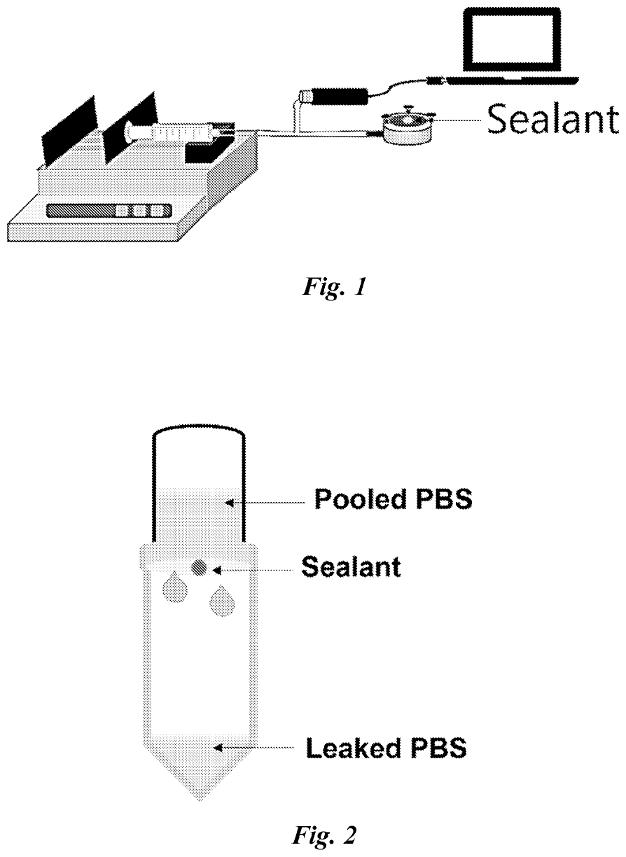

The burst pressure of the sealants was tested on porcine tissue substrates using an in house developed burst pressure test setup which is based on a slightly modified version of ASTM standard (F2392-04). Standard Test Method for Burst Strength of Surgical Sealants, ASTM International Website (publicly available; last accessed Aug. 16, 2023). The tissue substrates were cut into dimensions of approximately 2.5 cm by 2.5 cm. A puncture of about 3 mm diameter was made at the center of the tissue substrates using a biopsy punch. The substrates with sealant were then loaded onto the test fixture (FIG. 1). A syringe pump was used to pump test fluid into the test fixture at a rate of 5 ml/minute. A pressure transducer (PX409-100GUSBH, OMEGA Engineering, Norwalk, CT) connected to a computer was used to record the bursting pressures.

Initial studies to determine the best sealant formulations were performed on porcine skin tissues using 20 μl of the sealant. The sealant was allowed for cure for 2 hours in a humidifier at 37° C. After having identified the best formulations, 40 μl of sealant was applied to study the burst pressure strengths on all tissue substrates. The curing was carried out for 30 minutes and 2 hours in a in a humidifier at 37° C. For 30 minutes of the cure time, the samples were cured in humidifier for 20 minutes and then allowed to equilibrate at room temperature for 10 minutes. For the remaining 2 hours of cure time, the samples were cured in humidifier for 1.5 hours and then allowed to equilibrate at room temperature for remaining 30 minutes.

For all initial studies with all interfacial crosslinkers, 20 μl of the interfacial crosslinker solution was first applied on the tissues followed by application of 20 μl of the sealant. After identifying the best interfacial crosslinker, further studies were carried out by applying 40 μl of the crosslinker followed by application of 40 μl of the sealant. For all tissues the test fluid used was PBS except for stomach and lung tissue, in which case simulated gastric fluid and air were used, respectively.

### Example 4

### Effect of Ethanol to Water Ratio on Lap Shear Strength

Zein is only partially soluble in water or ethanol; however, a combination of water and ethanol in an appropriate ratio can completely dissolve zein. The ethanol to water ratio was varied to determine its effect on final adhesive strength of one of the sealants (Z40T7).

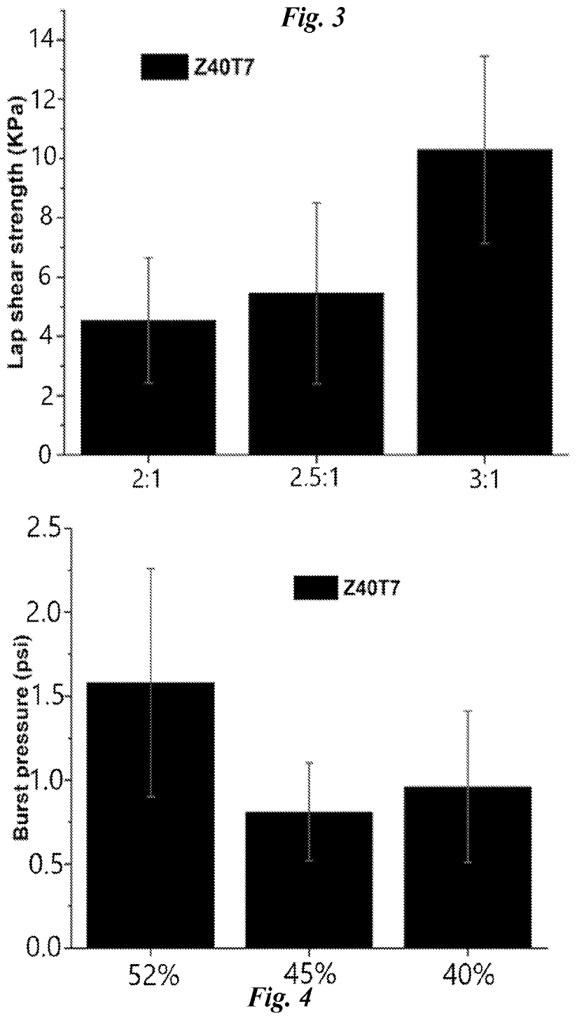

FIG. 3 shows the lap shear strength of sealant Z40T7 at three different ethanol to water ratios of 2:1, 2.5:1 and 3:1 which were 4.54±2.12 KPa, 5.45±3.05 KPa and 10.29±3.16 KPa, respectively. Adhesive failure was observed at all ratios tested. The adhesive strength increased with an increase in ethanol concentration. Accordingly, an ethanol to water ratio of 3:1 was used all further studies.

### Example 5

### Effect of Zein and Tannic Acid Concentration on Burst Pressure Strength

The concentration of zein, tannic acid (TA), ethanol, and water was chosen based on previous studies. To determine optimal zein and TA concentrations for the sealants, different sealant formulations were tested with zein concentration varied as 40 wt. %, 30 wt. % and 26 wt. %, while the TA concentrations were varied from 1-28 wt. % for each of the zein concentrations.

The range of TA concentrations studied for each zein concentration was limited by the final viscosity and consistency of the sealant. If the final consistency after mixing all the components was deemed highly viscous (i.e., could not be pipetted) or having a water-like consistency, no further studies were carried out with that particular formulation.

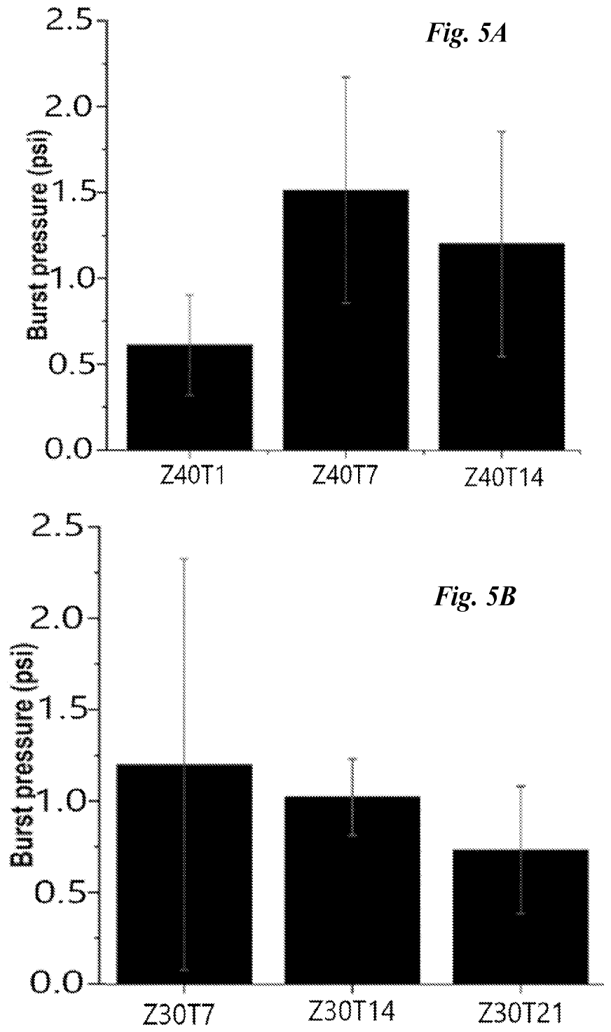

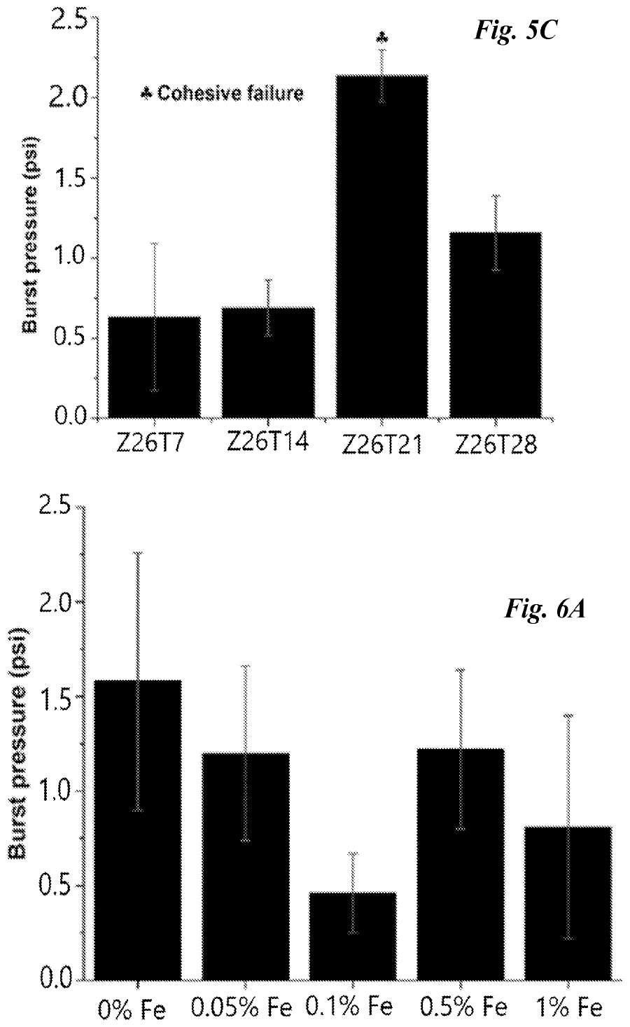

As shown in FIG. 5A, at a zein concentration of 40 wt. %, the burst pressure strength initially scaled with TA concentration. The highest value obtained for 7 wt. % TA was at 1.58±0.68 psi and the lowest value obtained for 1 wt. % TA was at 0.64±0.30 psi. However, upon further increasing the TA concentration to 14 wt. %, the burst pressure strengths dropped to 1.25±0.68 psi. In all cases, the sealants exhibited adhesive failure.

At a zein concentration of 30 wt. %, the burst pressure values decreased with an increase in TA concentration (FIG. 5B). The highest value was obtained for 7 wt. % TA at 1.21±1.13 psi, while burst pressure values decreased to 1.03±0.21 psi and 0.74±0.35 psi, respectively, upon decreasing the TA concentration to 14 wt. % and 21 wt. %. In all cases, the sealants exhibited adhesive failure.

As shown in FIG. 5C, at a zein concentration of 26 wt. %, the burst pressure values obtained for 7 wt. %, 14 wt. %, 21 wt. % and 28 wt. % TA were 0.63±0.46 psi, 0.69±0.17 psi, 2.14±0.16 psi and 1.16±0.23 psi respectively. Formulations with 21 wt. % TA not only had the highest burst pressure strengths, but the mechanism of failure was also cohesive in nature. All the other formulations exhibited adhesive failure.

Two formulations, Z40T7 (zein 40 wt. %, TA 7 wt. %) and Z26T21 (zein 26 wt. %, TA 21 wt. %) were chosen for further studies as they exhibited the best performance of the formulations tested in the burst pressure experiments.

### Example 6

### Effect of Adding Iron Crosslinker on Burst Pressure Strength

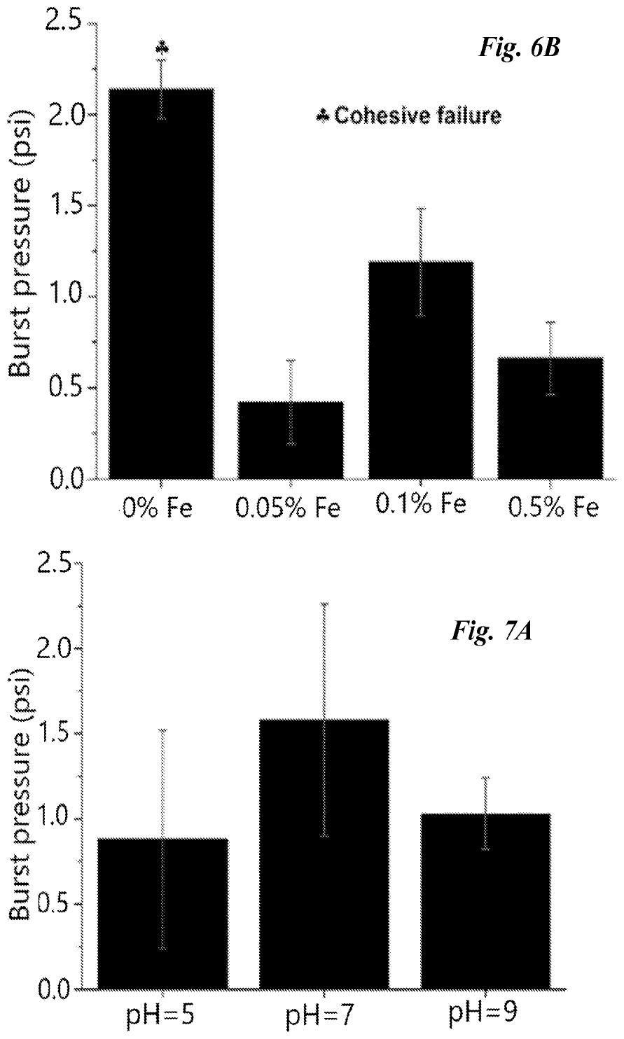

Transition metals like iron (Fe) are known to form complexes with catechol and oxidize them into their quinone form for further covalent crosslinking with substrates. FIG. 6A shows the effect of the addition of Fe crosslinkers to sealant Z40T7 at concentrations ranging from 0.05 wt. % to 1 wt. % on the burst pressure strength of the sealant. The burst pressure strength of Z40T7 incorporated with 0.05 wt. %, 0.1 wt. %, 0.5 wt. % and 1 wt. % Fe crosslinker was 1.20±0.46 psi, 0.46±0.21 psi, 1.22±0.42 and 0.81±0.59 psi, respectively. These values are lower than that for the formulation without any Fe crosslinker (1.58±0.68 psi).

FIG. 6B shows the effect of the addition of Fe crosslinker to sealant Z26T21 at concentrations ranging from 0.05 wt. % to 0.5 wt. %. The burst pressure strength of sealant Z26T21 incorporated with 0.05 wt. %, 0.1 wt. % and 0.5 wt. % of Fe crosslinker was 0.42±0.23 psi, 1.19±0.29 psi, and 0.66±0.19 psi, respectively. These results were similar to those obtained for Z40T7, with no significant improvement in burst pressure values observed when compared to the Z26T21 formulation with no Fe crosslinker (2.14±0.16 psi).

The addition of even the smallest amount of Fe crosslinker to the adhesive formulation led to a decrease in burst pressure strength of the sealant. All sealants incorporated with Fe crosslinker exhibited adhesive failure.

### Example 7

### Effect of pH on Burst Pressure Strength

From the formulations studied, those exhibiting the highest burst pressure strengths (e.g., Z40T7, Z26T21, Z40T7+0.5 wt. % Fe, and Z26T21+0.1 wt. % Fe) were studied across three pH values of 5, 7 and 9 to assess the effect of pH on final burst pressure strength of the sealant.

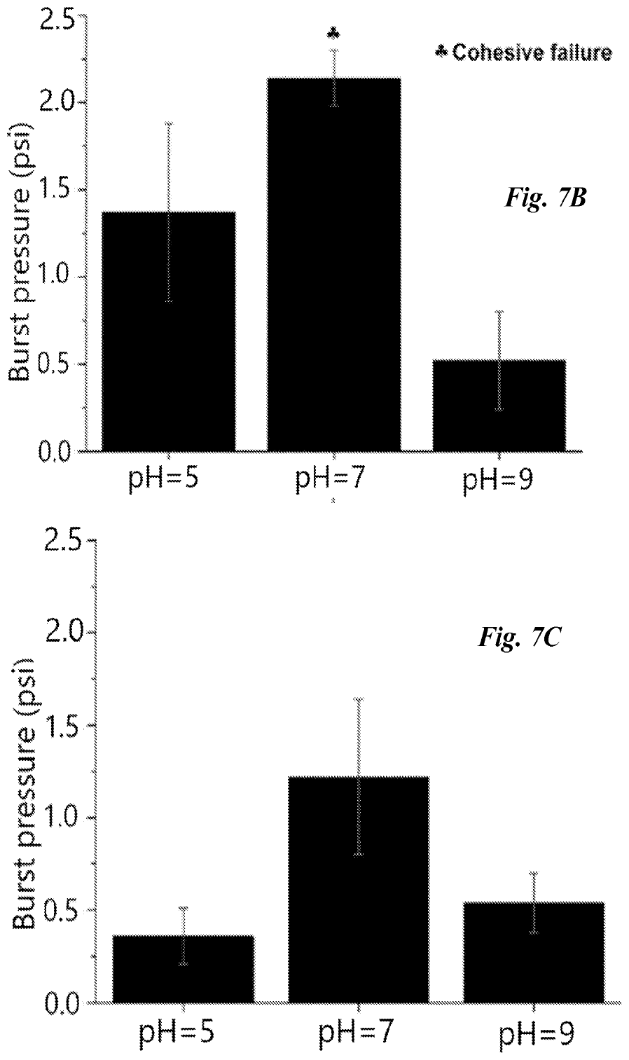

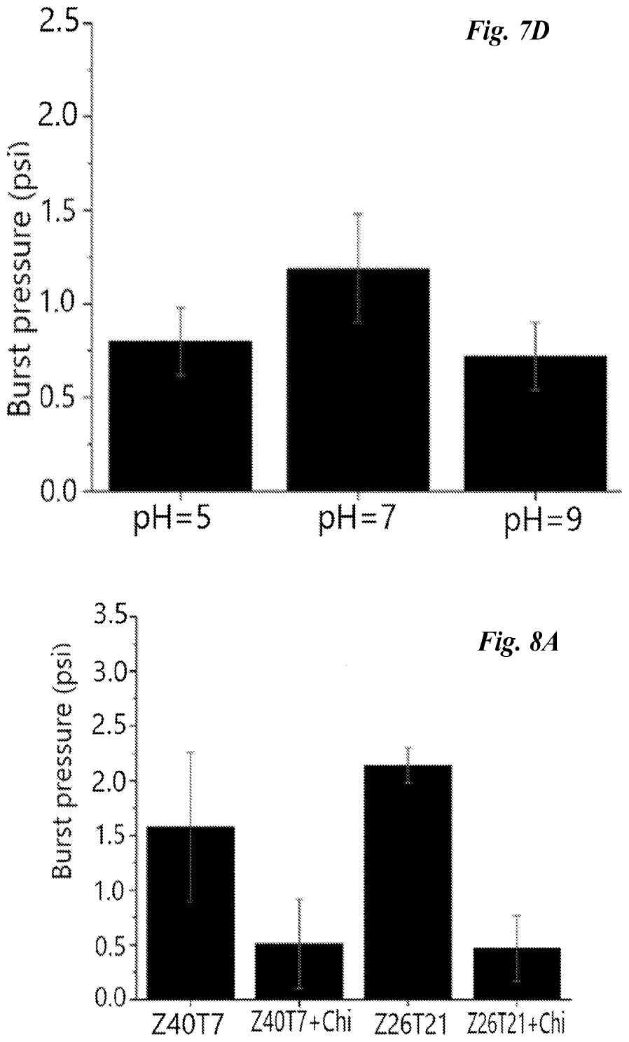

For Z40T7, the burst pressure strengths at a pH of 5, 7 and 9 were 0.88±0.64 psi, 1.58±0.68 psi, and 1.03±0.21 psi, respectively (FIG. 7A). For Z26T21, the burst pressure strength at a pH of 5, 7, and 9 were 1.37±0.51 psi, 2.14±0.16 psi, and 0.52±0.28 psi, respectively (FIG. 7B). Z40T7 exhibited adhesive failure at all pH values; however, Z26T21 exhibited cohesive failure at neutral pH and adhesive failure at all other pH values.

When 0.5 wt. % Fe crosslinker was added to Z40T7, the burst pressure strength at pH 5, 7, and 9 were 0.36±0.15 psi, 1.22±0.42 psi, and 0.54±0.16 psi, respectively (FIG. 7C). When 0.1 wt. % Fe crosslinker was added to Z40T7, the burst pressure strength at pH 5, 7, and 9 were 0.80±0.18 psi, 1.19±0.29 psi, and 0.72±0.18 psi, respectively (FIG. 7D). Both sealants exhibited adhesive failure at all pH values on incorporation of Fe crosslinker.

All the formulations performed optimally at a neutral pH (pH=7), which coincidentally is the physiological pH and supports the sealants developed in this study can be expected to perform with similar efficiency in vivo.

### Example 8

### Effect of Introducing Interfacial Crosslinker on Burst Pressure Strength

To further improve the burst pressures of the sealants, three interfacial crosslinkers were studied. Each of the three crosslinkers were chosen rationally and differed in their mechanism of crosslinking with the tissue and sealant. Initially, Chitosan was chosen due to its cationic nature.

Cheung et al., Chitosan: An Update on Potential Biomedical and Pharmaceutical Applications. Marine Drugs 13(8): 5156-5186 (2015). The tissues in body are anionic because of the anionic proteoglycans present in them. It was expected that Chitosan would enhance the adhesion strength through electrostatic interactions with the tissue and the sealant.

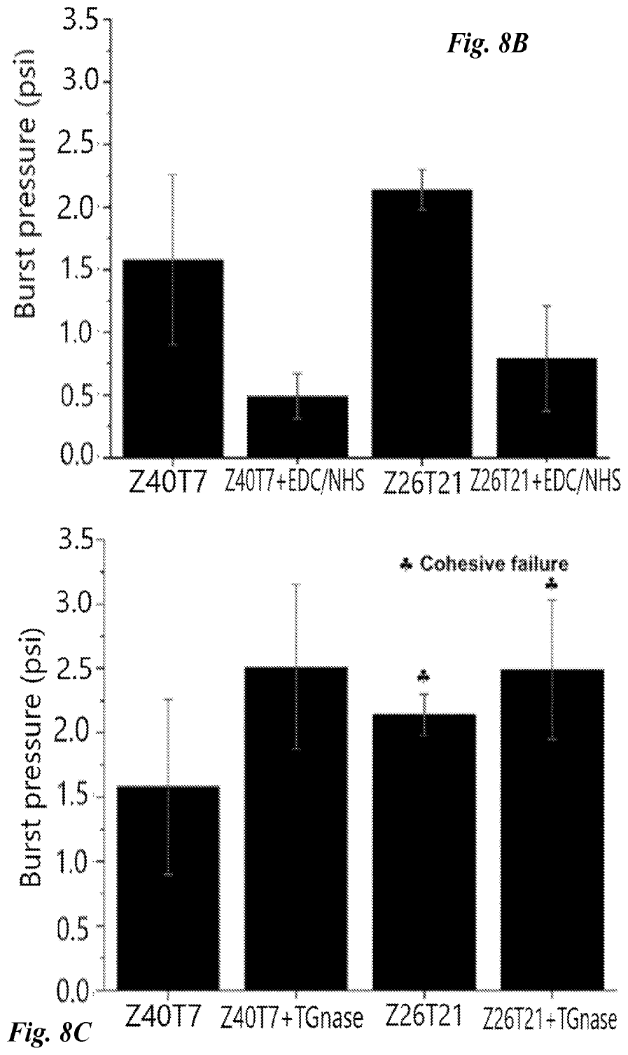

A 10 mg/ml viscous solution of Chitosan was prepared, and the pH was adjusted to 6 using acetic acid. As shown in FIG. 8A, using Chitosan as a crosslinker led to a decrease in the burst pressure of Z40T7 from 1.58±0.68 psi to 0.51±0.41 psi, as well as a decrease in the burst pressure of Z26T21 from 2.14±0.16 psi to 0.47±0.30 psi. All samples exhibited adhesive failure.

Further, carbodiimide chemistry using ethyl-3-(3-dimethylaminopropyl)carbodiimide hydrochloride (EDC) and N-hydroxy succinimide (NHS) was investigated as an interfacial crosslinker. EDC/NHS is known to be a zero length crosslinker (i.e., it does not incorporate into the macromolecule after crosslinking) and it has been widely used in tissue engineering applications. As such, its inclusion in the sealant formulation was hypothesized to facilitate the formation of amide bonds between the various carboxyl and hydroxyl functional groups of the zein-based sealant and tissues.

A 20 mg/ml solution of EDC/NHS (equal parts wt./wt.) was used for the study. As shown in FIG. 8B, EDC/NHS did not promote crosslinking of the sealant to the tissue. The burst pressure strength of Z40T7 decreased from 1.58±0.68 psi to 0.49±0.18 psi, and the burst pressure strength of Z26T21 decreased from 2.14±0.16 psi to 0.79±0.42 psi. All samples exhibited adhesive failure.

Finally, commercial food-grade transglutaminase (TGnase) enzyme was investigated as a potential interfacial crosslinker. TGnase is known to help catalyze the formation of isopeptide bonds between γ-carboxamide groups of glutamines and the ε-amino groups of lysine. TGnase has been widely used to bond different proteins together and to bond proteins to non-protein molecules with required functional groups like hyaluronic acid. Here it was hypothesized that TGnase would help bond proteins in zein to proteins and functional groups on tissues.

A viscous solution of TGnase was prepared by mixing 500 mg of transglutaminase with 1200 μl of deionized water (DI) and the prepared solution was used immediately. FIG. 8C supports that using TGnase as the crosslinker led to an improvement in the burst pressure of Z40T7 from 1.58±0.68 psi to 2.51±0.64 psi, and an improvement in the burst pressure of Z26T21 from 2.14±0.16 psi to 2.49±0.54 psi.

Z26T21 (which exhibited cohesive failure when used on its own) exhibited cohesive failure even when TGnase was used as an interfacial crosslinker. Further, all samples with Z40T7 and Z40T7+TGnase exhibited adhesive failure. Accordingly, TGnase was determined to be the interfacial crosslinker of choice and was used in all further studies.

### Example 9

### Ex-Vivo Studies: Porcine Stomach and Sausage Casing

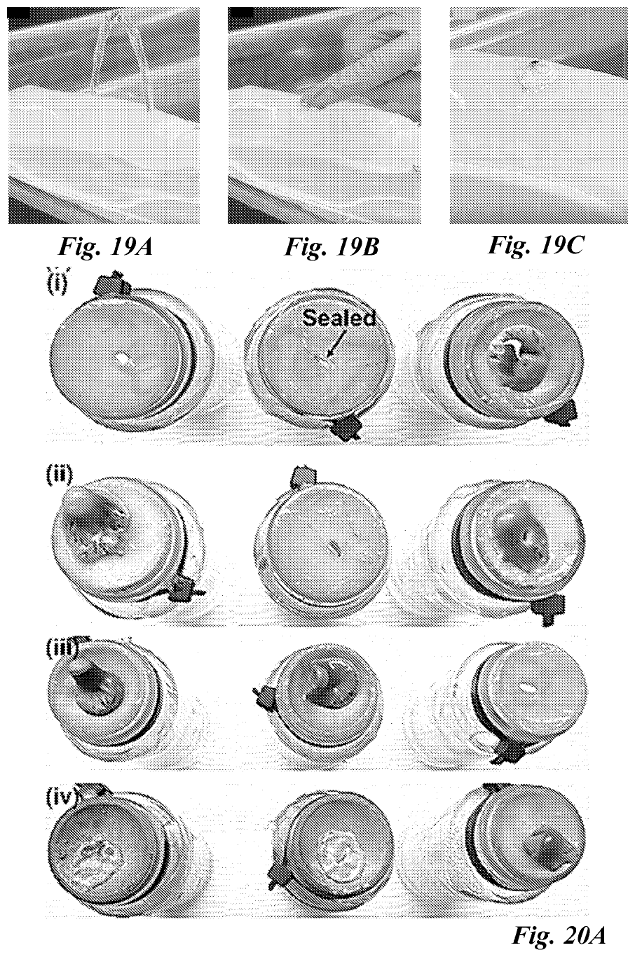

In the body, body fluids and stomach contents can interfere with the sealing and curing process. Accordingly, ex vivo experiments were performed using porcine stomach tissue (FIG. 18) and sausage casing (FIG. 19) to demonstrate the efficacy of the sealant in plugging leaking punctures in tissue. It is understood that sausage casing is prepared from the small intestine and, thus, responds similarly therewith.

A stationary ex vivo model was designed to study the tissue sealing ability of the sealants under physiological conditions. Briefly, porcine stomach was filled with water-colored with red dye. The stomach was then placed under water, a hole was punctured using a 3 mm biopsy punch, and the dyed water was allowed to flow through the puncture. The sealant was filled into a syringe and incubated at 70° C. for 24 hours to partially remove some solvent. The sealant was then applied to the puncture on the stomach underwater. After the puncture was sealed, the stomach tissue was carefully transferred to a new jar of water to test any leakage from the sealed puncture.

As shown in FIG. 18, when a 3 mm puncture was introduced onto the stomach tissue, dyed water leaked from the puncture. Upon sealing the puncture with Z40T7 under wet conditions underwater, an instant seal was formed, and no further leak was detected. Z26T21 also sealed the puncture instantly underwater. Sealant with TGnase could not be studied in this case because TGnase instantly dissolves underwater.

The ability of the sealants to seal actively leaking punctures under pressure was also tested using an ex vivo flow model. A flow model was designed using sausage casing whereby water flowed continuously through it at a pressure of 0.6 psi (higher than normal physiological pressure in intestines). More specifically, sausage casing was cut into appropriate length and connected to a water pump which circulated water continuously through the sausage casing at a pressure of 0.6 psi. As shown in FIGS. 19A-19C, a 3 mm puncture was made using a biopsy punch (FIG. 19A). Prepared glue Z40T7 was applied onto the leaking puncture for 10 seconds with the application of mild pressure (FIG. 19B). The change in pressure before and after sealing the puncture was noted.

Upon application of the adhesive Z40T7, the puncture instantly stopped leaking and was sealed (FIG. 19C). The seal was still intact even after 1 hour.

Z26T21, Z26T21+TGnase, and Z40T7+TGnase were also tested pursuant to this methodology (i.e., applied on the leaking puncture for 10 seconds with the application of mild pressure). Z26T21+TGnase and Z40T7+TGnase were not able to seal the puncture instantly.

### Example 10

### Ex-Vivo Studies: Long Term Adhesion Study in Wet Environment

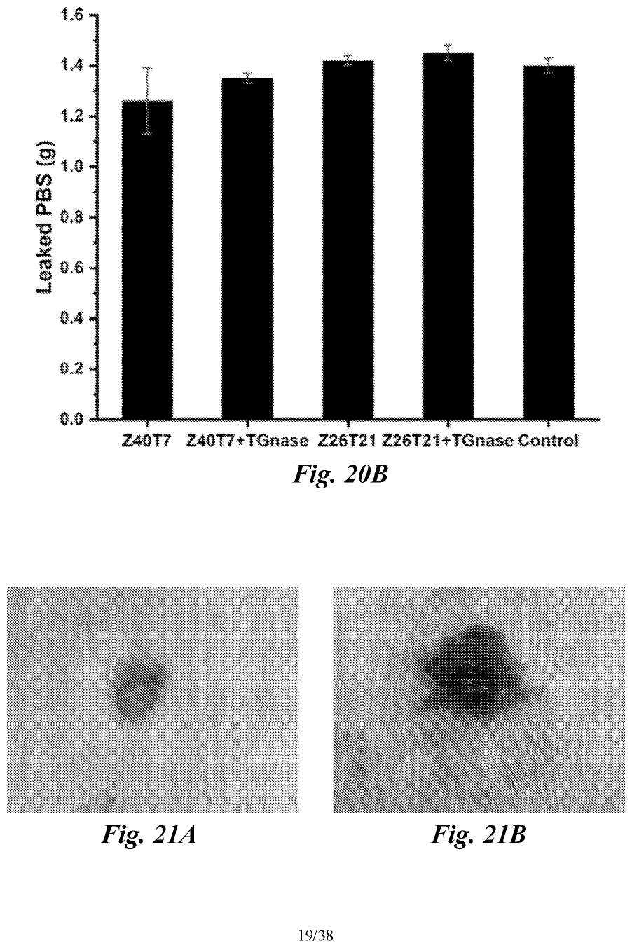

Tissue sealant must seal the tissue and prevent any body fluid leakage from the tissues at least until new tissue starts to regenerate. Accordingly, the ability of the sealants to remain in contact with the tissues over extended periods in presence of body fluids was examined (FIG. 2). Briefly, sausage casing was applied to the mouth of a 20 ml vial filled with 4 ml of PBS using a zip tie. A 3 mm hole was punctured and 100 μl of each of the sealant was applied to seal the puncture. The vials were then inverted such that the PBS pooled on top of the sealant and sausage casing, and it was mounted on top of a 50 ml Eppendorf tube of known weight (FIG. 20A). The entire assembly was enclosed inside a Styrofoam box and was placed on a shaker at 40 rpm in the incubator at 37° C. A moist environment was created within the Styrofoam box using a large container filled with water. The assembly was removed after 72 hours and the weight of liquid collected in the Eppendorf tube was analyzed. A control experiment was performed without a puncture and sealant on the sausage casing.

After 72 hours, 2 out of 3 replicates of adhesive Z40T7, 2 out of 3 replicates of adhesive Z40T7+TGnase and adhesive Z26T21, and all three replicates of adhesive Z26T21+TGnase remained in contact with the tissues (FIG. 20B). Sausage casing being permeable caused some of the PBS to leak into the container below. Therefore, a control experiment was designed with no puncture or sealant to determine if the amount of PBS permeating from sausage casing with no puncture was similar to those which were punctured and sealed with the sealant. Only those samples whereby sealants remained intact even after 72 hours were included in the calculations. It was found that the amount of leaked PBS collected after 72 hours in the samples in which the sealant remained intact was similar to that of the control experiment.

### Example 11

### Ex-Vivo Studies: Tissue Sealing Ability on Topical Wounds

The tissue sealing ability of the sealant was also study via ex vivo full thickness skin defect model on rat cadavers to evaluate the efficacy of the sealant on topical wounds on skin. The hair on the backs of euthanized rats (used for a different study) were shaved off using a trimmer. A 3 mm puncture was made on the skin of each cadaver (FIG. 21A). Z40T7 and Z26T21 sealants were each pre-cured at 70° C. within a 3 ml syringe for 24 hours. The prepared sealant was dispensed onto the injury site using a 18G blunt tip needle (FIG. 21B shows Z40T7 applied onto the puncture). The sealant was allowed to dry for 5 minutes.

Sealant Z40T7 adhered strongly to the skin and residual hair on the rat skin and did not come off even when the skin was tugged and pulled using a tweezer.

### Example 12

### Burst Pressure Strength on Different Tissue Substrates

### Skin

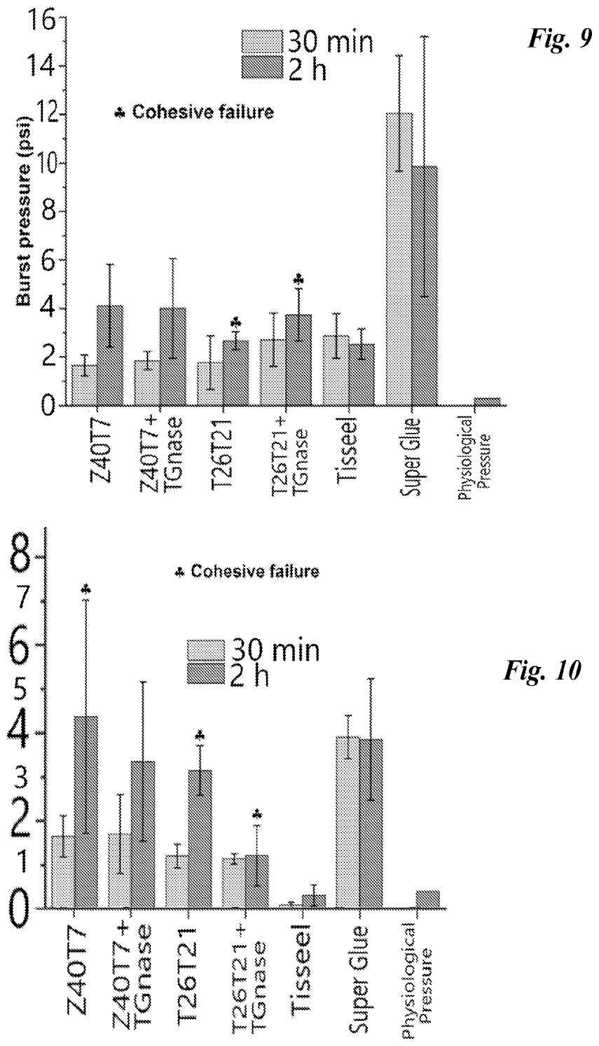

FIG. 9 shows the burst pressures of all the zein-based tissue sealants compared against commercially available Fibrin based tissue sealant (Tisseel) and cyanoacrylate glue (Superglue) at two different cure times of 30 minutes and 2 hours on porcine skin tissues. The plot also shows the normal physiological pressure experienced in the abdomen.

The burst pressures of Z40T7 increased from 1.66±0.43 psi to 4.12±1.71 psi when the cure time increased from 30 minutes to 2 hours. Similarly, for Z40T7+TGnase, the burst pressures for 30 minutes and 2-hour cure times were 1.85±0.37 psi and 4.01±2.06 psi, respectively. The burst pressures for Z26T21 at 30 minutes and 2-hour cure times were 1.77±1.11 psi and 2.67±0.37 psi, respectively. The burst pressures for Z26T21+TGnase at 30 minutes and 2-hour cure times were 2.71±1.1 psi and 3.74±1.07 psi, respectively. By comparison, the burst pressures for Tisseel at 30 minutes and 2-hour cure times were 2.87±0.92 psi and 2.53±0.62 psi, respectively, and the burst pressures for Superglue at 30 minutes and 2-hour cure times were 12.04±2.39 psi and 9.85±5.36 psi, respectively.

As shown in FIG. 9, all zein-based sealants tested performed better than Tisseel. Out of all the sealants tested, only Z26T21 and Z26T21+TGnase exhibited cohesive type failure at 2-hour cure time. Moreover, the normal pressure experienced in a human abdomen is about 0.29 psi, which is much less than the values for the zein-based sealants studied, even at a short cure time of 30 minutes. Rooban et al., Comparing Intra-Abdominal Pressures in Different Body Positions via a Urinary Catheter and Nasogastric Tube: A Pilot Study, Annals Intensive Care 2(Suppl 1): 1-7. (2012); Hamoud et al., Gastric and Urinary Bladder Pressures Correlate with Intra-Abdominal Pressure in Patients with Morbid Obesity, J Clinical Monitoring & Computing 36: 1021-1028 (2022); de Keulenaer et al., What Is Normal Intra-Abdominal Pressure and How Is It Affected by Positioning, Body Mass and Positive End-Expiratory Pressure?, Intensive Care Medicine 35(6): 969-976 (2009). The highest burst pressure obtained among the zein-based adhesives was for Z40T7.

### Sausage Casing

FIG. 10 shows the burst pressure data of all sealants studied on sausage casing at two different cure times. The burst pressures of Z40T7 at 30 minutes and 2-hour cure times were 1.63±0.47 psi and 4.35±2.65 psi, respectively. The burst pressures for Z40T7+TGnase at 30 minutes and 2-hour cure times were 1.68±0.9 psi and 3.33±1.81 psi, respectively. The burst pressures for Z26T21 at 30 minutes and 2-hour cure times were 1.18±0.27 psi and 3.13±0.57 psi, respectively. The burst pressures for Z26T21+TGnase at 30 minutes and 2-hour cure times were 1.12±0.12 psi and 1.19±0.69 psi, respectively. The burst pressures for Tisseel at 30 minutes and 2-hour cure times were 0.07±0.06 psi and 0.29±0.24 psi, respectively. The burst pressures for Superglue at 30 minutes and 2-hour cure times were 3.89±0.49 psi and 3.83±1.38 psi, respectively.

All zein-based sealants tested performed better than Tisseel on sausage casing at both cure times. Out of the sealants tested, three of the zein-based adhesives (i.e., Z40T7, Z26T21 and Z26T21+TGnase) exhibited cohesive type failure at 2-hour cure time. Moreover, the normal pressure experienced in human small intestines is about 0.38 psi, which is much less than the values for the zein-based sealants tested even at a short cure time of 30 minutes. Scott et al., The Nocturnal Jejunal Migrating Motor Complex: Defining Normal Ranges by Study of 51 Healthy Adult Volunteers and Meta-Analysis, Neurogastroenterology & Motility 18(10): 927-935 (2006). The highest burst pressure obtained among the zein-based adhesives was for Z40T7.

### Heart

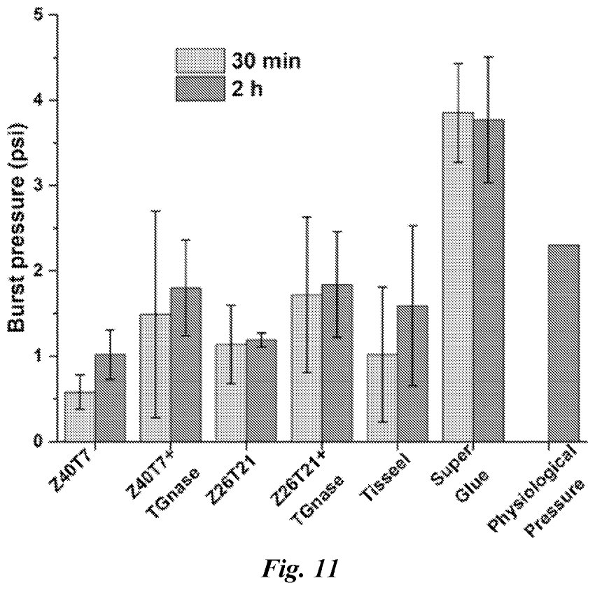

FIG. 11 shows the burst pressure data of the zein-based tissue sealants on porcine heart tissues at two different cure times. The burst pressures of Z40T7 at 30 minutes and 2-hour times were 0.58±0.20 psi and 1.02±0.29 psi, respectively. The burst pressures for Z40T7+TGnase at 30 minutes and 2-hour cure times were 1.49±1.21 psi and 1.8±0.56 psi, respectively. The burst pressures for Z26T21 at 30 minutes and 2-hour cure times were 1.14±0.46 psi and 1.19±0.08 psi, respectively. The burst pressures for Z26T21+TGnase at 30 minutes and 2-hour cure times were 1.72±0.91 psi and 1.84±0.62 psi, respectively. The burst pressures for Tisseel at 30 minutes and 2-hour cure times were 1.02±0.79 psi and 1.59±0.94 psi, respectively. The burst pressures for Superglue at 30 minutes and 2-hour cure times were 3.85±0.58 psi and 3.77±0.74 psi, respectively.

All zein-based sealants tested performed better than Tisseel on heart tissue at both cure times. All the sealants exhibited adhesive failure. Moreover, the highest pressure experienced in a human heart is the peak systolic pressure in left ventricle and brachial artery which is about 2.30 psi and is higher than the values for the zein based sealants and commercial fibrin sealant Tisseel. Reichek & Devereux, Reliable Estimation of Peak Left Ventricular Systolic Pressure by M-Mode Echographic-Determined End-Diastolic Relative Wall Thickness: Identification of Severe Valvular Aortic Stenosis in Adult Patients, Am Heart J 103(2): 202-209 (1982). The highest burst pressure was obtained for Z26T21+TGnase.

### Stomach

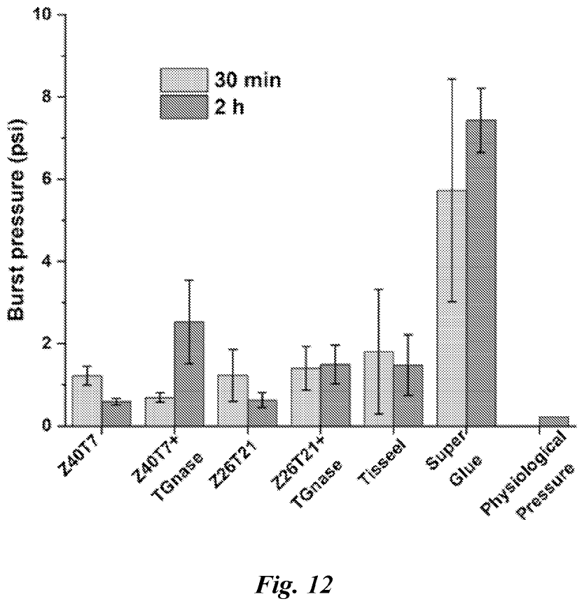

FIG. 12 shows the burst pressure data of the zein-based tissue sealants on porcine stomach tissue at two different cure times. The burst pressures of Z40T7 at 30 minutes and 2-hour cure times were 1.22±0.23 psi and 0.59±0.08 psi, respectively. The burst pressures for Z40T7+TGnase at 330 minutes and 2-hour cure times were 0.69±0.11 psi and 2.53±1.01 psi, respectively. The burst pressures for Z26T21 at 30 minutes and 2-hour cure times were 1.23±0.63 psi and 0.63±0.18 psi, respectively. The burst pressures for Z26T21+TGnase at 30 minutes and 2-hour cure times were 1.4±0.53 and 1.49±0.47 psi, respectively. The burst pressures for Tisseel at 30 minutes and 2-hour cure times were 1.81±1.51 psi and 1.48±0.74 psi, respectively. The burst pressures for Superglue at 30 minutes and 2-hour cure times were 5.73±2.71 psi and 7.43±0.78 psi, respectively. Adhesive failure was observed for all the sealants. Moreover, the normal pressure experienced in a human stomach is about 0.23 psi, which is much less than the values for the zein-based sealants tested even at a short cure time of 30 minutes. The highest burst pressure was obtained for Z40T7+TGnase. Rooban et al. (2012), supra; Hamoud et al. (2021), supra.

### Lungs

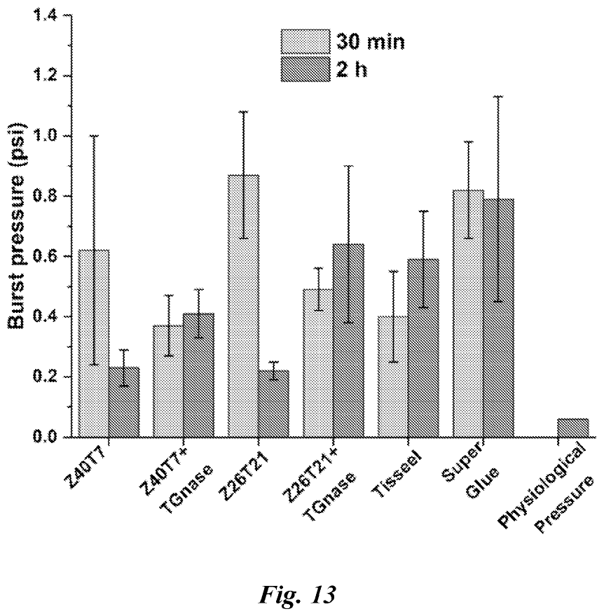

FIG. 13 shows the burst pressure data of the zein-based tissue sealants on porcine lung tissues at two different cure times. The burst pressures of Z40T7 at 30 minutes and 2-hour cure times were 0.62±0.38 psi and 0.23±0.06 psi, respectively. The burst pressures for Z40T7+TGnase at 30 minutes and 2-hour cure times were 0.37±0.1 psi and 0.41±0.08 psi, respectively. The burst pressures for Z26T21 at 30 minutes and 2-hour cure times were 0.87±0.21 psi and 0.22±0.03 psi, respectively. The burst pressures for Z26T21+TGnase at 30 minutes and 2-hour cure times were 0.49±0.07 and 0.64±0.26 psi, respectively. The burst pressures for Tisseel at 30 minutes and 2-hour cure times were 0.4±0.15 psi and 0.59±0.16 psi, respectively. The burst pressures for Superglue at 30 minutes and 2-hour cure times were 0.82±0.16 psi and 0.79±0.34 psi, respectively.

All zein-based sealants tested performed better than Tisseel on lung tissue at both cure times. Adhesive failure was observed for all the sealants. Moreover, the normal pressure experienced in human lungs is about 0.06 psi which is much less than the values for the zein-based sealants tested even at a short cure time of 30 minutes. Zielinska-Krawczyk et al., Pleural Manometry—Historical Background, Rationale for Use and Methods of Measurement, Respiratory Medicine 136: 21-28 (2018). The highest burst pressure was obtained for Z26T21+TGnase.

### Liver

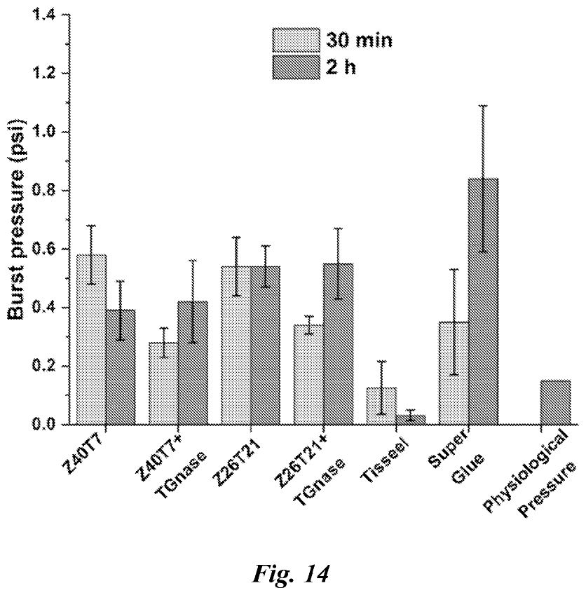

FIG. 14 shows the burst pressure data of the zein-based tissue sealants on liver at two different cure times. The burst pressures of Z40T7 at 30 minutes and 2-hour cure times were 0.58±0.1 psi and 0.39±0.1 psi, respectively. The burst pressures for Z40T7+TGnase at 30 minutes and 2-hour cure times were 0.28±0.05 psi and 0.42±0.14 psi, respectively. The burst pressures for Z26T21 at 30 minutes and 2-hour cure times were 0.54±0.10 psi and 0.54±0.07 psi, respectively. The burst pressures for Z26T21+TGnase at 30 minutes and 2-hour cure times were 0.34±0.03 psi and 0.55±0.12 psi, respectively. The burst pressures for Tisseel at 30 minutes and 2-hour cure times were 0.13±0.09 psi and 0.03±0.02 psi, respectively. The burst pressures for Superglue at 30 minutes and 2-hour cure times were 0.35±0.18 psi and 0.84±0.25 psi, respectively.

All zein-based sealants tested performed better than Tisseel on liver tissues at both cure times. Adhesive failure was observed for all the sealants. The liver being a well vascularized organ, has blood flow from hepatic and portal veins and arteries. 75%-80% of blood supply to the liver is through the portal veins which carries partially deoxygenated blood that leaves the stomach, spleen, large and small intestine, pancreas, and gallbladder. The remaining 25% of blood flow can be attributed to the hepatic artery which carries well-oxygenated blood.

The blood pressure in the hepatic arteries of a human is similar to that of normal blood pressure of 2.3 psi which is higher than the burst pressure reported here for any of the sealants. However, the blood pressure in portal veins is lower at about 0.15 psi, which is less than the values for the zein-based sealants tested even at a short cure time of 30 minutes. Eipel et al., Regulation of Hepatic Blood Flow: The Hepatic Arterial Buffer Response Revisited, World J Gastroenterology: WJG 16(48): 6046-6057 (2010); Blumgart et al., Liver Blood Flow: Physiology, Measurement, and Clinical Relevance, Surgery Liver, Biliary Tract & Pancreas 1: 37-53 (2007).

The highest burst pressure was obtained for Z40T7.

### Aorta

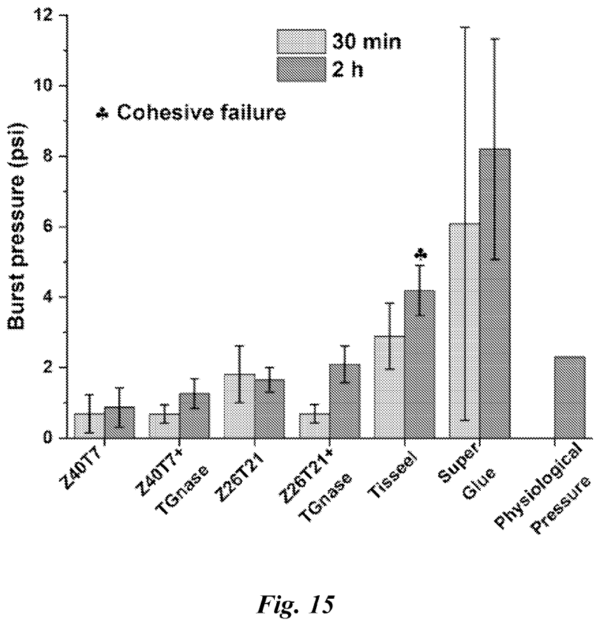

FIG. 15 shows the burst pressure data of the zein-based tissue sealants on an aorta at two different cure times. The burst pressures of Z40T7 at 30 minutes and 2-hour cure times were 0.69±0.54 psi and 0.87±0.56 psi, respectively. The burst pressures for Z40T7+TGnase at 30 minutes and 2-hour cure times were 0.68±0.26 psi and 1.26±0.42 psi, respectively. The burst pressures for Z26T21 at 30 minutes and 2-hour cure times were 1.81±0.81 psi and 1.65±0.35 psi, respectively. The burst pressures for Z26T21+TGnase at 30 minutes and 2-hour cure times were 0.69±0.26 psi and 2.09±0.52 psi, respectively. The burst pressures for Tisseel at 30 minutes and 2-hour cure times were 2.89±0.94 psi and 4.19±0.71 psi, respectively. The burst pressures for Superglue at 30 minutes and 2-hour cure times were 6.08±5.58 psi and 8.2±3.13 psi, respectively.

Tisseel performed better than all the zein-based sealants tested on aorta tissue at both cure times. All the sealants exhibited adhesive type failure except for Tisseel, which exhibited cohesive failure. Moreover, the normal pressure experienced in a human aorta is about 2.3 psi which is higher than the values for the zein-based sealants tested. Homan et al., Physiology, Pulse Pressure. StatPearls (2021). The highest burst pressure was obtained for Z40T7.

### Dura Mater

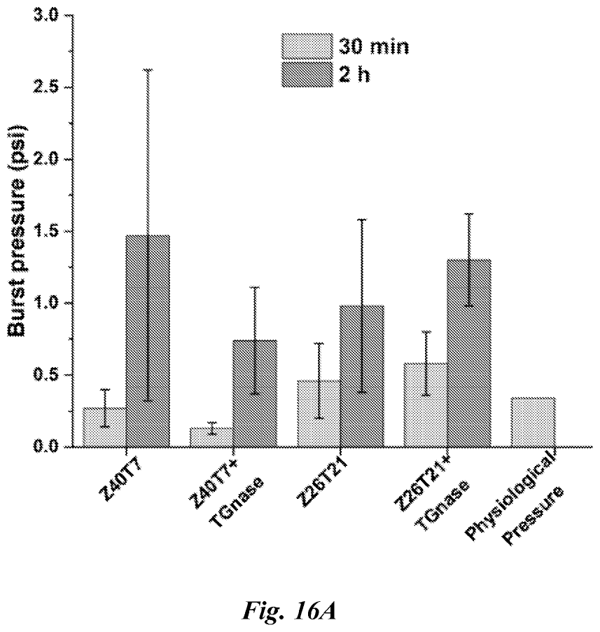

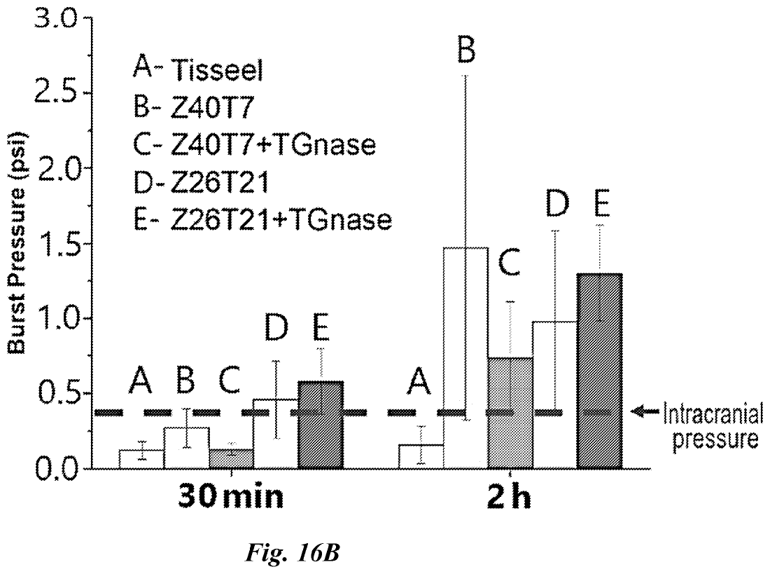

FIGS. 16A and 16B show the burst pressure data of all zein-based tissue sealants on dura mater at two different cure times. The burst pressures of Z40T7 at 30 minutes and 2-hour cure times were 0.27±0.13 psi and 1.47±1.15 psi, respectively. The burst pressures for Z40T7+TGnase at 30 minutes and 2-hour cure times were 0.13±0.04 psi and 0.74±0.37 psi, respectively. The burst pressures for Z26T21 at 30 minutes and 2-hour cure times were 0.46±0.26 psi and 0.98±0.6 psi, respectively. The burst pressures for Z26T21+TGnase at 30 minutes and 2-hour cure times were 0.58±0.22 psi and 1.3±0.32 psi, respectively.

All zein-based sealants exhibited adhesive type failure. Moreover, the normal pressure experienced in dura is about 0.34 psi, which is higher than the values for the zein-based sealants tested. Rangel-Castillo et al. (2008), supra. The highest burst pressure was obtained for Z40T7.

In sum, the zein-based sealants did not perform similarly on all tissue substrates. Moreover, each of the four sealants performed best on different tissue substrates. This supports that the adhesion between a sealant and tissue of interest is greatly affected by the composition of the sealant and the tissue itself. Another essential consideration is matching the mechanical properties like elastic modulus or stiffness of the sealant with underlying tissues.

For a short cure time of 30 minutes, the highest burst pressures on skin, sausage casing, stomach, heart, lungs, liver, aorta, dura, and small intestine were exhibited by Z26T21+TGnase, Z40T7, Z26T21+TGnase, Z26T21+TGnase, Z26T21+TGnase, Z40T7, Z26T21, Z26T21+TGnase, and Z40T7, respectively. For a 2-hour cure time, the highest burst pressures on skin, sausage casing, stomach, heart, lungs, liver, aorta, dura, and small intestine were exhibited by Z40T7, Z40T7, Z40T7+TGnase, Z26T21+TGnase, Z26T21+TGnase, Z26T21+TGnase, Z26T21+TGnase, Z40T7, and Z26T21, respectively. In most cases, the sealant that exhibited the highest burst pressures at a 30-minute cure time (i.e., when the sealant is partially cured) was not the one that exhibited the highest burst pressures at complete curing (i.e., at 2-hours). This could be due to the change in interactions over longer cure times between the sealant and tissues, or due to a change in the mechanical properties of the sealants with curing time.

Moreover, in case of some tissues like stomach, lungs, and liver, the burst pressures of some sealants at 30-minute cure time were higher than that for 2-hour cure time. This could be attributed to mismatch in mechanical properties of sealants with that of tissue after complete curing at 2 hours. Such mechanical disparity between the sealants and the underlying tissue can lead to mechanical stress that is concentrated at the tissue-sealant interface which could disrupt the chemical and physical links between them leading to delamination or failure.

### Example 13

### Lap Shear Strength on Porcine Tissue Substrates

The lap shear strength of all zein-based sealants was tested on porcine tissue substrates across two different cure times of 30 minutes and 2 hours. The lap shear strength of Z40T7 at 30 minutes and 2 hours were 5.11±2.75 KPa and 31.38±13.74 KPa, respectively. The lap shear strength of Z40T7+TGnase at 30 minutes and 2 hours were 12.37±1.75 KPa and 40.85±13.92 KPa, respectively. The lap shear strength of Z26T21 at 30 minutes and 2 hours were 8.22±0.31 KPa and 35.45±7.32 KPa, respectively. The lap shear strength of Z26T21+TGnase at 30 minutes and 2 hours were 14.28±3.16 KPa and 46.5±15.94 KPa, respectively.

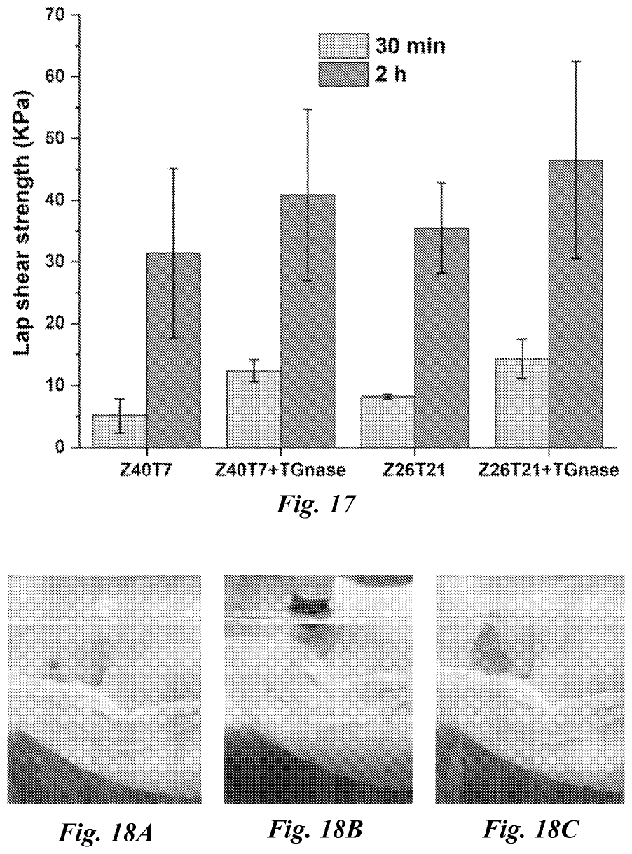

Adhesive failure was observed for all sealants. The highest lap shear adhesion strength values on porcine skin tissue substrates were obtained for Z26T21+TGnase, although the highest burst pressures were obtained for Z40T7 (see FIG. 17).

### Example 14

### Comparison of Zein-Based Sealants with Commercial Tisseel and Super Glue

It was observed that the zein-based sealants performed better than Tisseel for all tissue substrates except the aortic tissue (in which case Tisseel performed far better than all zein-based sealants). Tisseel, a fibrin-based sealant, is a two-component sealant that involves the combining of purified fibrinogen and thrombin in presence of calcium ions and factor XIII, or a transglutaminase. The activated factor XIII or TGnase catalyzes amide formation between the γ-carboxamide of glutamine and ε-amino groups of lysine on the fibrin polymer glutamine (formed by thrombin activated conversion of fibrinogen to fibrin monomer and subsequent self-assembly into fibrin polymer via hydrogen bonding) and tissues.

It is speculated that the reason why Tisseel performed better on aortic tissues as compared to any other tissues studied could be due to a higher availability of all components contributing towards fibrin sealant formation in aorta tissues (which inherently carries blood in the body).

Super Glue, a cyanoacrylate-based sealant, performed better than all of the zein-based sealants and Tisseel. Due to the high electronegativity of the alkoxy carbonyl and nitrile groups on cyanoacrylate monomers, the unsaturated carbon double bonds on the acrylate monomer are electronically polarized and undergoes Michael addition reactions with mild bases such as water and amines on tissues. Although these provide strong adhesion to most tissues, the mechanical disparity between this extremely stiff sealant and the soft underlying tissues poses the risk of tissue damage. Furthermore, Super Glue also breaks down into cytotoxic products including formaldehyde. For at least these reasons, Super Glue does not have widespread application in the medical/surgical arts.

Moreover, it was interesting to note that the trend of burst pressures across various tissues was rather comparable across all the sealants studied, including Tisseel and Super Glue. For example, for softer tissues (such as liver and lung tissues), even a strong adhesive like Super Glue exhibited lower burst pressure values (similar to all other sealants tested) when compared to burst pressure values on stiffer tissues like skin and stomach.

### Example 15

### Burst Pressure on Porcine Small Intestines

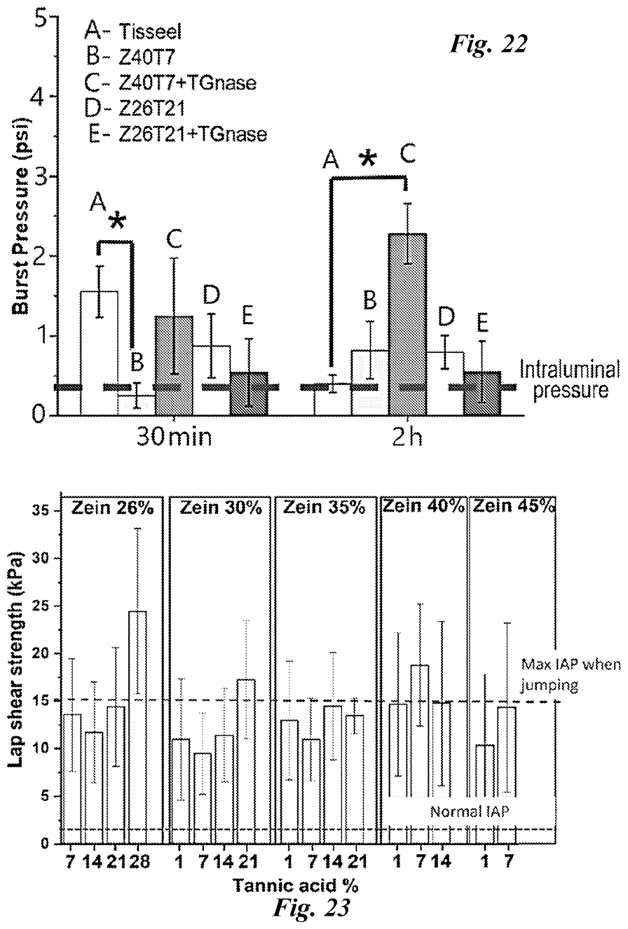

FIG. 22 shows the burst pressure data of all the sealants studied on porcine small intestines at two different cure times. The burst pressures of Z40T7 at 30 minute and 2-hour cure times were 0.25±0.15 psi and 0.82±0.36 psi, respectively. The burst pressures for Z40T7+TGnase at 30 minute and 2-hour cure times were 1.25±0.72 psi and 2.28±0.38 psi, respectively. The burst pressures for Z26T21 at 30 minute and 2-hour cure times were 0.87±0.39 psi and 0.79±0.21 psi, respectively. The burst pressures for Z26T21+TGnase at 30 minute and 2-hour cure times were 0.55±0.42 psi and 0.55±0.38 psi, respectively. The burst pressures for Tisseel at 30 minute and 2-hour cure times were 1.55±0.32 psi and 0.40±0.11 psi, respectively. Except for Z26T21 at the 30-minute cure time and Tisseel at both cure times which exhibited cohesive failure, all the other sealants exhibited adhesive type failure. Moreover, the normal pressure experienced in small intestines is about 0.38 psi which is higher than the values for the zein based sealants studied here. The highest burst pressure was obtained for Z40T7+TGnase.

### Example 16

### Further Lap Shear Studies on Various Zein and TA Ratios

To further determine optimal zein and TA concentrations for the sealants, lap shear testing was performed on various zein and TA ratios similar to the study of Example 5. Here, the zein percentage varied from 26-45 wt. % and TA percentage was varied from 1-28 wt. %. Formulations with low amounts of both zein and TA had low viscosity and no tackiness and, hence, were excluded from the study. Some formulations with high amounts of both zein and TA were again not studied due to extremely high viscosity and low tack.

Based on the above, seventeen formulations were identified and lap shear adhesion strengths were studied on porcine skin tissue. The adhesives were cured for 2 hours in a humid environment at 37° C. FIG. 23 shows the lap shear adhesion strengths of all the formulations tested. All the zein adhesives showed adhesion strengths higher than normal intra-abdominal pressures (IAP) experienced in humans. When 26 wt %, of zein was used, a TA loading of 28 wt. % resulted in highest lap shear strength. When 30 wt %, of zein was used, a TA loading of 21 wt. % resulted in highest lap shear strength. When 35 wt %, of zein was used, a TA loading of 14 wt. % resulted in highest lap shear strength. When 40 wt %, of zein was used, a TA loading of 7 wt. % resulted in highest lap shear strength. When 45 wt %, of zein was used, a TA loading of 7 wt. % resulted in highest lap shear strength. Overall, the highest lap shear strength was obtained when 26 wt. % zein was mixed with 28 wt. % TA.

The lap shear strength of an FDA-approved fibrin-based tissue sealant was also studied at two different cure times as shown in Table 1 below. The adhesion strengths at both cure times were not found to be statistically different than the adhesive formulations of the present disclosure that were tested.

TABLE 1
Lap shear strength of FDA-approved tissue
sealant (ARTISS) at different cure times

| | |
|---|---|
| Cure Time | Lap Shear Strength (kPa) |

| | | |
|---|---|---|
| 30 | minutes | 18.5 ± 8.09 |
| 2 | hours | 13.60 ± 5.39 |

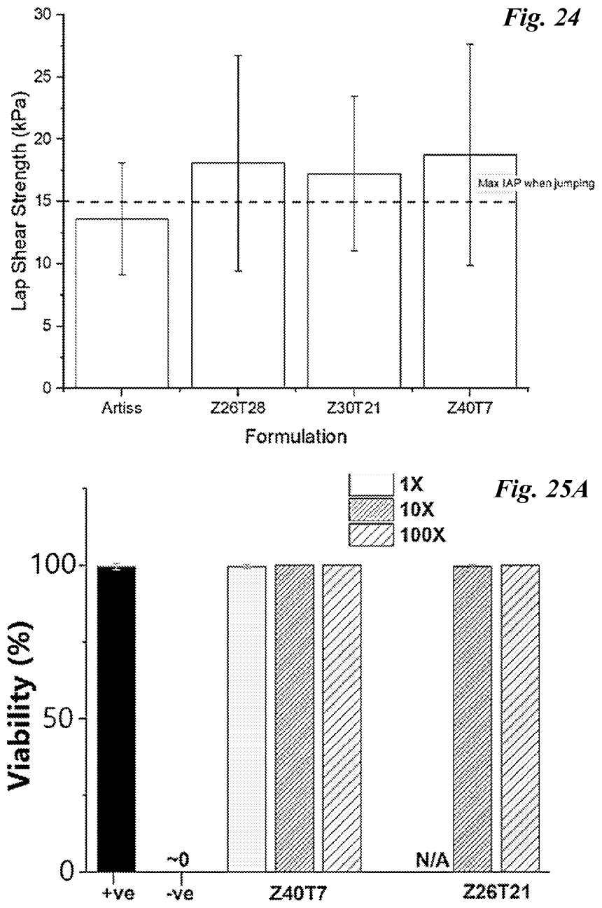

FIG. 24 shows the comparison of lap shear strength of ARTISS with the zein-based adhesives hereof at a 2 hour cure time. The adhesion strength of ARTISS was not only lower than at least formulations Z26T28, Z30T21 and Z40T7, it was also much lower than the maximum intra-abdominal pressure experienced by a human when jumping.

### Example 17

### Cytocompatibility Studies

NIH 3T3 fibroblasts cells were cultured in high-glucose Dulbecco's modified Eagle's medium (DMEM) with 10% fetal bovine serum (Lonza, Basel, Switzerland) and 1% penicillin/streptomycin antibiotic. Cells were subculture every 3 days or when confluency was ≥60%. The cytocompatibility of the zein-based sealants were studied by culturing the cells in sealant extracts in accordance with the ISO 10993-5 standard.

Briefly, 200 μl of sealants were cured in humid environment for 72 hours at 37° C. to form a cylindrical sample. The samples were then immersed in 5 ml of DMEM for 24 hours to obtain the leachate. The leachate was studied at three different dilutions of 1×, 10× and 100×. Leachate was sterile-filtered using a 0.2-μm filter. Cells were seeded onto PLL-coated coverslips at 5000 cells/cm² and incubated overnight. After cells were attached, the medium was replaced with 500 μl of leachate. Cells were incubated in leachate for 24 hours.

To measure viability, a live/dead assay was used. Cells were rinsed with PBS containing 0.01 wt % calcium chloride and 0.01 wt % magnesium chloride (PBS+) to prevent detachment. Negative control samples were incubated with 70% ethanol for 5 minutes at room temperature. Samples were stained with calcein-acetoxymethyl and ethidium homodimer (LIVE/DEAD Viability/Cytotoxicity kit, Thermo Fisher, Waltham, MA) for 30 minutes at 37° C., rinsed, and mounted onto microscope slides. Fluorescent images were obtained with an excitation of 495 nm and emission of 532 nm.

A PrestoBlue assay (Thermo Fisher, Waltham, MA) was used to assess metabolic activity. Cells on PLL-coated coverslips served as positive controls, and uncoated glass coverslips without cells were used as background controls. After incubation, triplicate aliquots were removed and placed into 96-well plates. The fluorescence of the reagent was measured using excitation at 555 nm and emission at 595 nm.

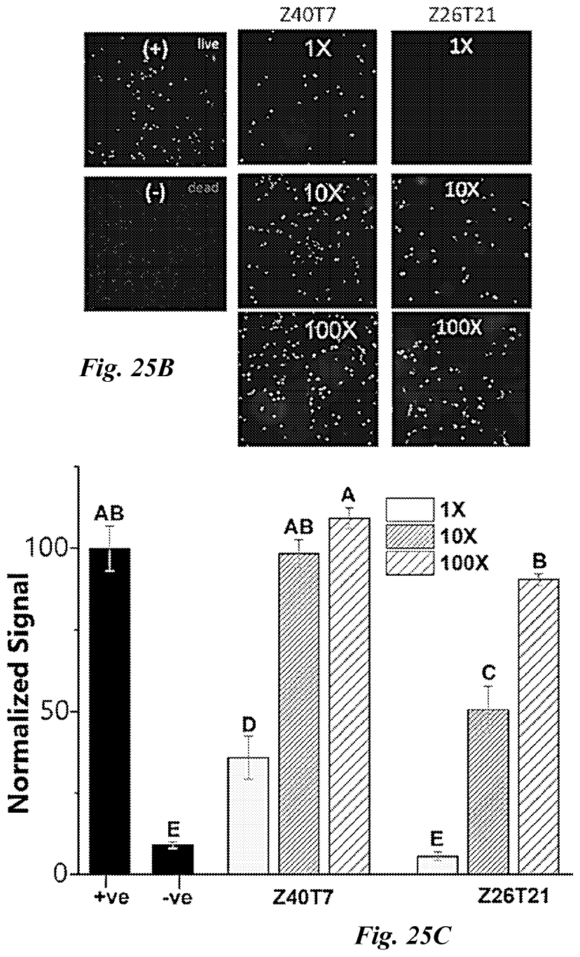

As shown in FIGS. 25A and 25B, Z40T7 showed cell viability at all leachate concentrations, while Z26T21 only exhibited viability when the leachate was diluted to 10× and 100×. Moreover, the results from the PrestoBlue assay shown in FIG. 25C support that cell metabolic activity increased for both Z40T7 and Z26T21 with increasing leachate dilution. The low cell viability and metabolic activity could be from the catechol groups in TA which cause reactive oxygen species (ROS) generation.

### Example 18

### Gamma Irradiation of Sealants for In Vivo Studies

For viable in vivo use of the sealants, it would be useful for the sealants to withstand sterilization. For example, to study the in vivo wound healing ability of these sealants, they must first be sterilized using a suitable method. Gamma irradiation was chosen as the sterilization method.

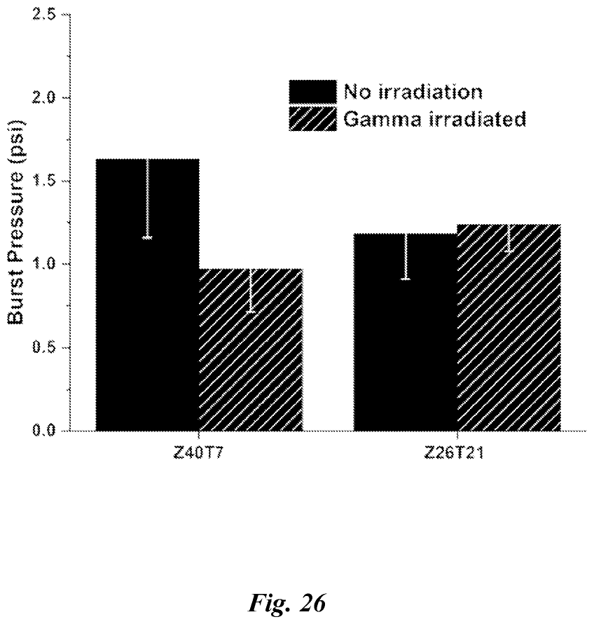

The sealants were exposed to gamma irradiation up to 30 kGy. The burst pressure of the sealants were then tested by curing them for 30 minutes cure time on sausage casings. The burst pressure Z40T7 slightly decreased from 1.63±0.47 psi to 0.97±0.26 psi (FIG. 26). However, for Z26T21, the burst pressure was very similar before and after gamma irradiation.

### Example 19

### In Vivo Rat Wound Healing Study

The in vivo wound healing ability of zein-based sealants was studied on male Sprague Dawley (SD) rat dorsal skin. The experiments consist of 4 test articles: Z40T7 sealant, Tisseel, suture, and Z40T7 sealant+suture. The wound healing in rats were studied at different time points of 7 days and 14 days with n=4 rats being studied for each time points.

Briefly, the hair from the dorsal section was removed to expose the skin. Four equally spaced 1 cm long incisions were made on the rat dorsal skin and each of the four test articles were applied onto each wound. The surgery was performed under general anesthesia (isoflurane). The weight and activity level of the rats were recorded initially and periodically during the experiment. The injury site was examined for signs of irritation, and photographs of the injury site was acquired on days 0, 4, 7, 10 and 14 to determine the wound closure rate. The rats were euthanized at the end of each time point of study. The tissues from the injury sites were harvested and further processed for histopathological analysis.

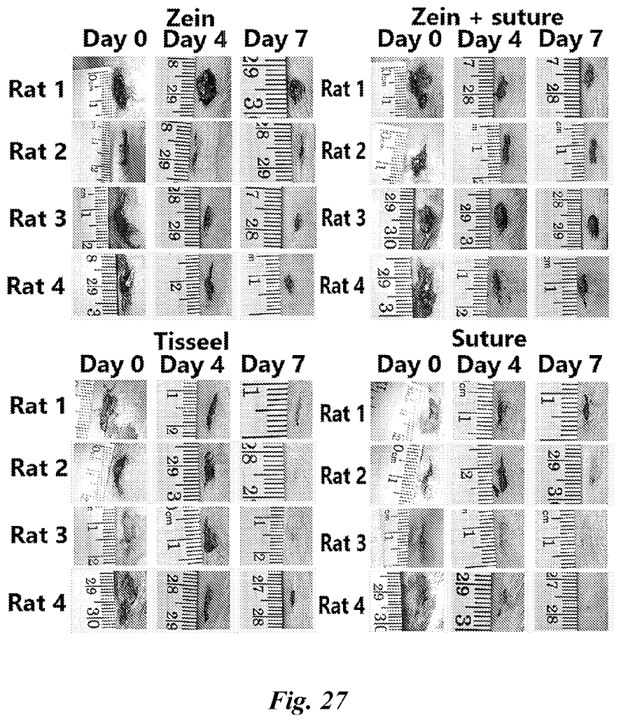

FIG. 27 shows the photographs of application of the four test articles on all rats in the 7-day study. In all cases the wound area decreased with time. Healing proceeded with formation of a scab around the wound in some cases and without any complications. Moreover, in for the Z40T7+suture group, the sealant remained adhered to the wound even at the end of day 7.

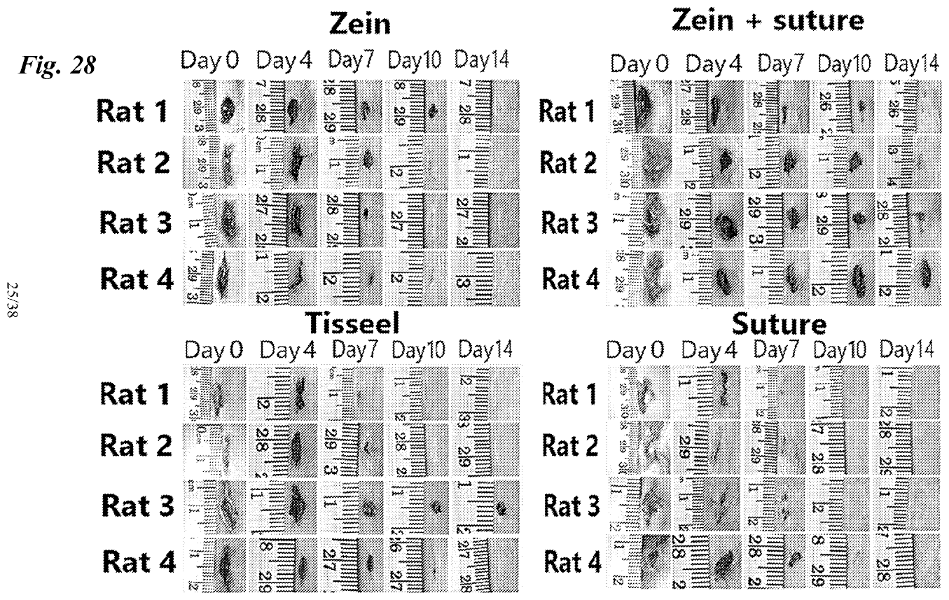

FIG. 28 shows the photographs of application of the four test articles on all rats in the 14-day study. In all cases the wound had almost completely healed by day 14. The suture only group showed complete wound healing by day 10, while the Z40T7 only group healed completely by day 14. In case of Tisseel only group, the wounds had healed for all mice except for one where some scabbing remained at day 14. The Z40T7+suture group also showed scabbing and bulging from injected adhesive.

### Example 20

### Feasibility of Zein-Based Adhesives for Use as a Tissue Adhesive for Sealing Punctures in Dura Mater in Combination with a Graft Material

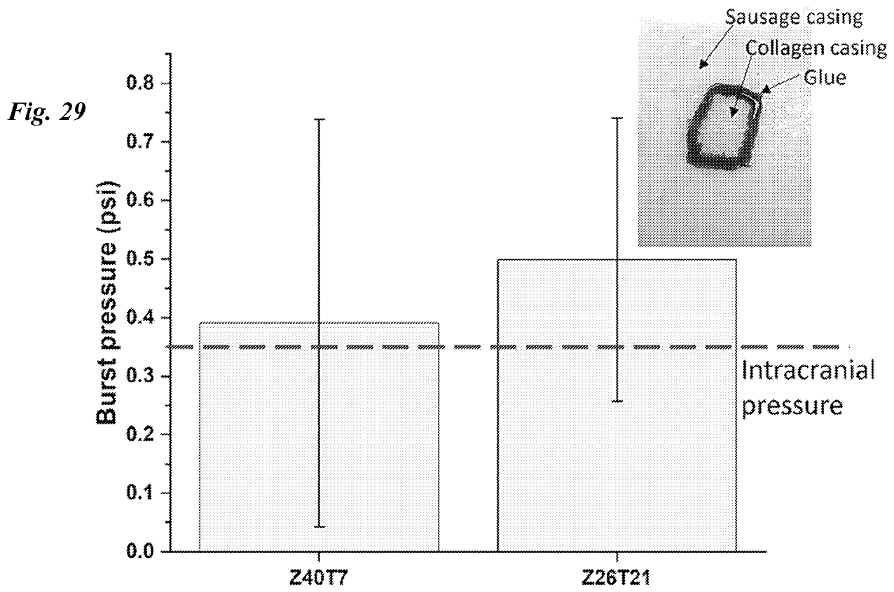

The adhesion strength of different zein-based adhesives when used as an adhesive to bond a graft material to dura mater was evaluated. Initially, sausage casings and collagen casings were used as model tissue and graft material respectively. A 1 cm puncture was made on the sausage casing. A 2*2 cm collagen casing was placed on top of the sausage casing and the edges were sealed using zein adhesives (Z40T7 and Z26T21). The adhesive was allowed to cure for 30 minutes after which burst pressure was studied. FIG. 29 shows the burst pressure of both adhesives, both of which were higher than the normal intracranial pressures of 0.34 psi.

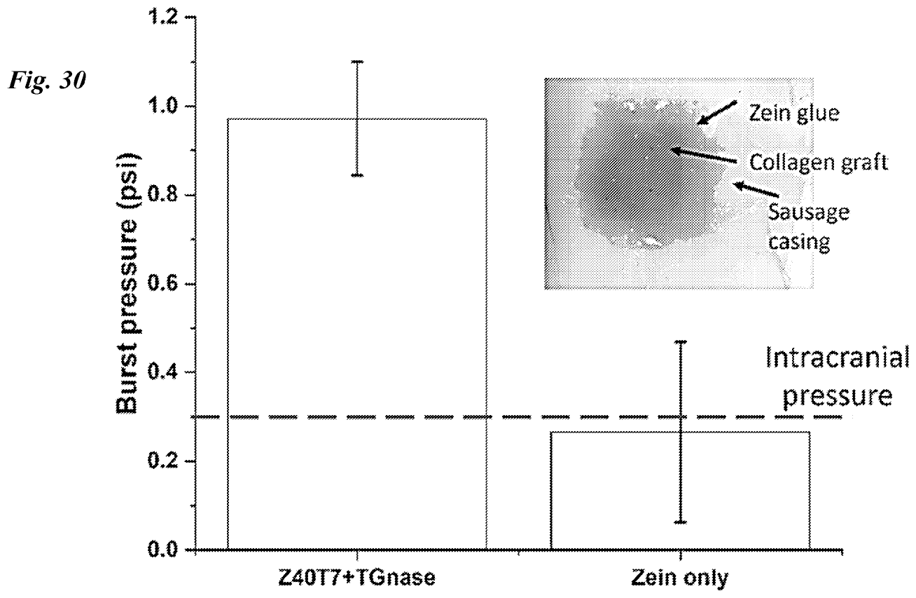

Thereafter, the adhesive application was modified to resemble application in real-time by applying the adhesive on top of the graft instead of only applying the adhesive on the edges. The cure time was also reduced to 15 minutes. Three zein formulations (Zein only (without TA), Z40T7 and Z26T21) were applied alone and in combination with transglutaminase powder (TGnase). Only Z40T7+TGnase showed adhesion strengths higher than intracranial pressure (FIG. 30).

Next, the burst pressures were evaluated to study adhesion strength when adhesive was applied on dura mater in combination with a commercial graft as compared against sausage casing with collagen casing graft. The adhesive was applied all over the graft material in each instance and allowed 15 minutes cure time.

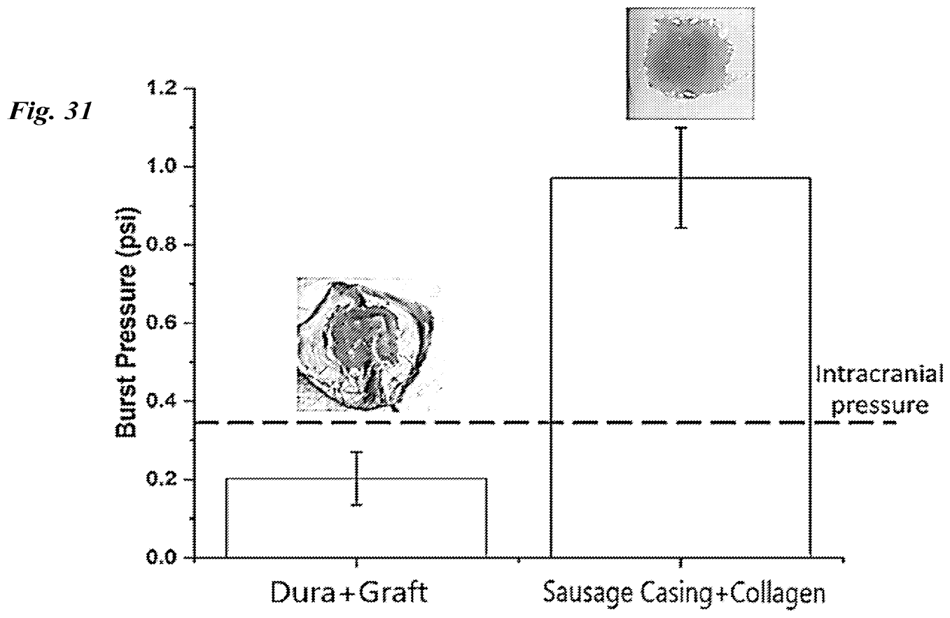

FIG. 31 shows the burst pressures of Z40T7+TGnase when applied on dura mater with graft. The burst pressures were much lower than the values obtained when sausage casing was glued to a collagen casing. The values were also lower than normal intracranial pressures.

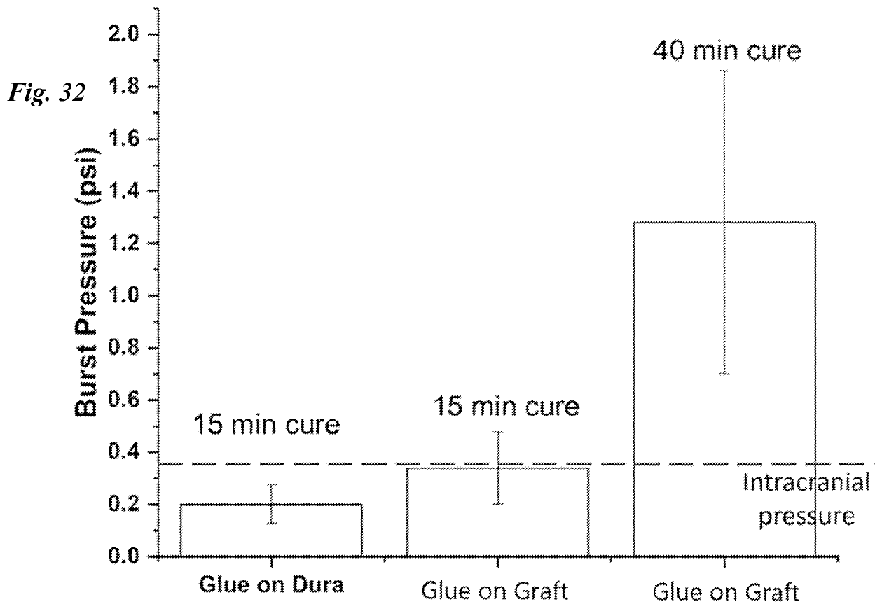

To standardize the tests and reduce variability in results from applications, the adhesives were then only be applied either directly on a 2*2.5 cm sized graft and pressured onto the 1 cm puncture on dura or the adhesives can be applied directly in a 2*2.5 cm area around the puncture and the same sized piece of graft can be applied on top of the adhesive. About 80 μl of adhesive was used. As shown in FIG. 32, the adhesion strength was higher when adhesive was applied first on the graft for a 15-minute cure time. The adhesion strength increased further when cure time was increased to 40 minutes.

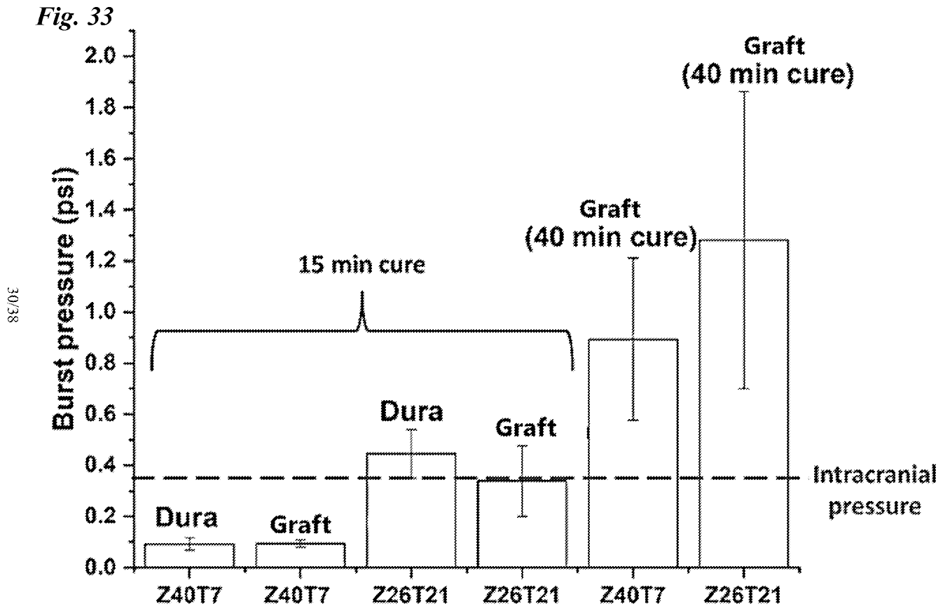

The adhesive volume was increased from 80 μl to 150 μl when applied on dura as shown in FIG. 33. This caused the adhesion strength to increase especially for Z26T21 even at a 15-minute cure time. The adhesive volume when applied on the graft was kept the same at 80 μl. The adhesion strengths for Z26T21 were higher than intracranial pressures.

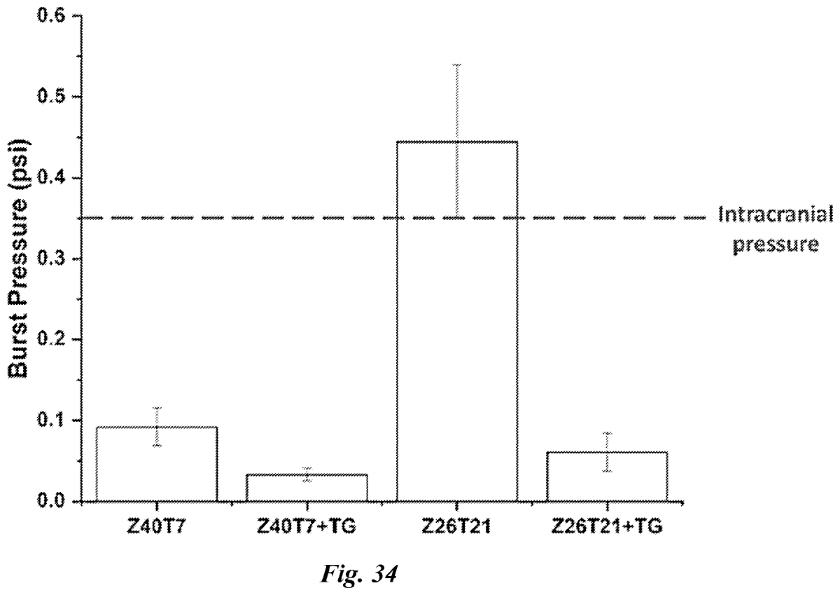

The effect of transglutaminase incorporation on burst pressure was also studied. 150 μl of transglutaminase solution was first applied onto the dura followed by application of 150 μl of zein-based adhesive. The cure time was 15 minutes at room temperature. The adhesion strengths decreased tremendously when transglutaminase was applied (FIG. 34).

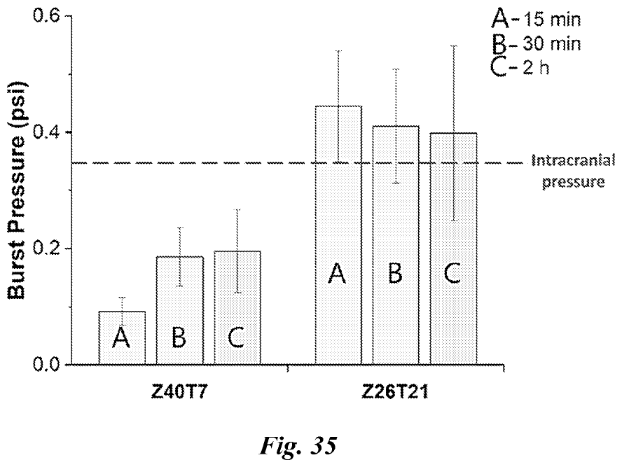

The cure time was increased from 15 minutes to 30 minutes and 2 hours as shown in FIG. 35. For Z40T7, the burst pressures increase from 0.09 psi at 15 minutes to 0.18 psi and 0.19 psi at 30 minutes and 2 hours, respectively. For Z26T21, the burst pressures did not show any appreciable change with increase in cure time; however, all values for Z26T21 were higher than intracranial pressures which was not the case for Z40T7.

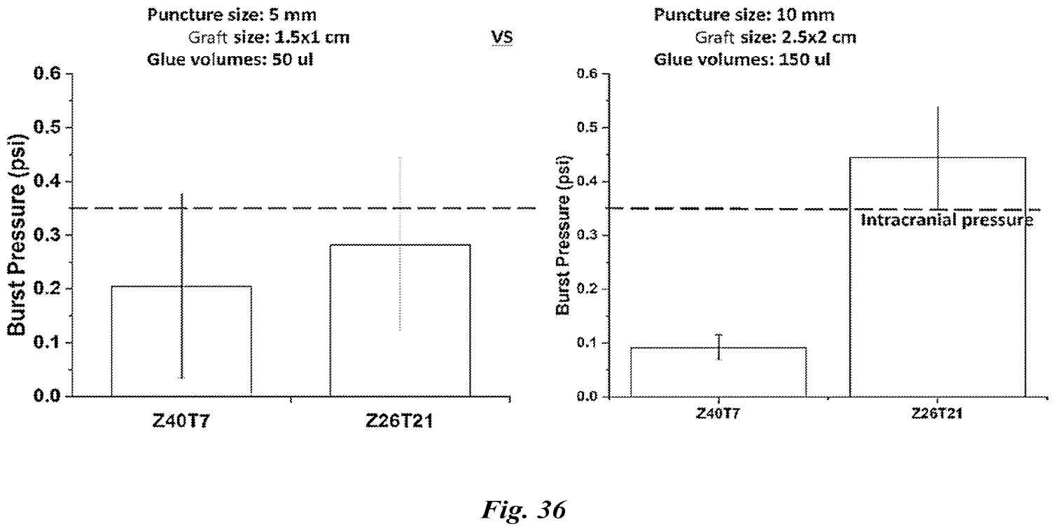

Next, the effect of reducing puncture size from 10 mm to 5 mm was evaluated. The size of the graft used as an on lay graft was also reduced accordingly as shown in FIG. 36. The burst pressures decreased when the puncture sizes reduced which was contrary to what was expected. Therefore, it was hypothesized that the puncture size does not play a huge role in burst pressures strengths as long as the bonding area between dura and the graft is sufficiently high.

The lap shear adhesion strengths of bonding dura to the graft were also evaluated at different cure times. Dura and grafts were cut into 1.5*1.2 cm sections and bonded to aluminum substrates using super glue. 30 μl of Z40T7 or Z26T21 was applied to dura and the grafts, which were overlapped together to form a lap-shear joint. A 50 g lead weight was placed on top to improve contact between both overlapping surfaces.

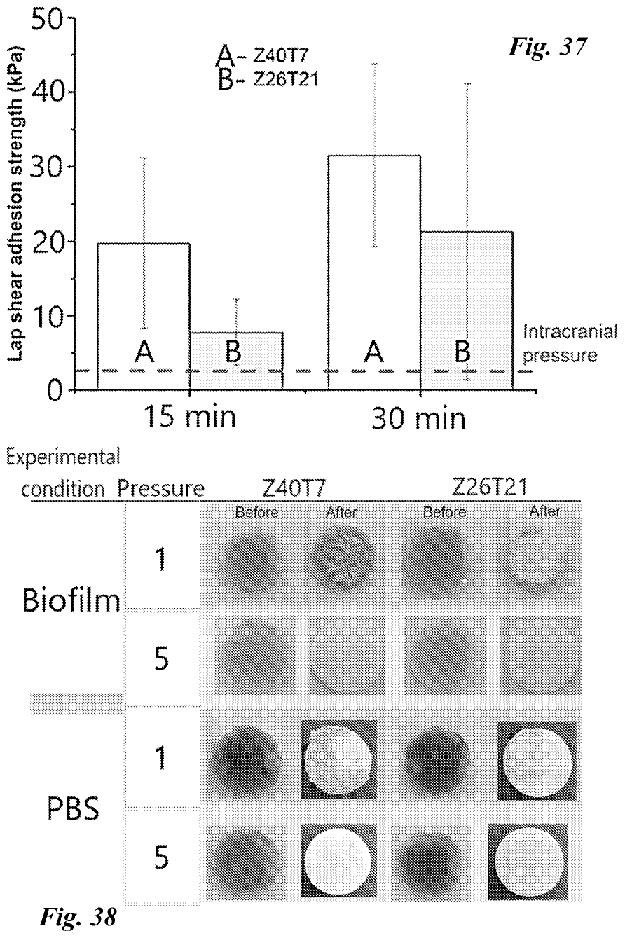

The adhesion strength of Z40T7 increased from 19.73±11.43 psi at 15 minutes to 31.53±12.23 psi at 30 minutes. The adhesion strength of Z26T21 increased from 7.77±4.49 psi at 15 minutes to 21.29±19.92 psi at 30 minutes. The highest adhesion strength was obtained for Z40T7 as shown in FIG. 37, although the highest burst pressures were recorded for Z26T21.

### Example 21

### Adhesion Studies on Hydroxyapatite

The adhesion strength of Z40T7 and Z26T21 formulations were further studied on hydroxyapatite (HAP) substrates to check its feasibility for applications within an oral environment. HAP substrates were immersed in PBS for 5 minutes or saliva (to form a biofilm). 20 μl of the adhesive was coated onto the HAP tiles using a spatula. The adhesive was allowed to cure for 10 minutes in ambient conditions. A commercial water flosser (Waterpik Ultra) was rastered on the coatings for 1 minute to study the adhesion of coatings. The water flosser tip was set at a constant height of 1 mm from the substrate. The images of the substrate before and after the water flosser tests were captured to analyze the amount of adhesive remaining on the substrates after the test. FIG. 38 shows some representative images of the adhesive coatings on HAP substrates before and after water flosser tests. It was observed that adhesion to HAP was stronger for Z40T7 compared to Z26T21. The adhesion strengths seemed to be similar regardless of whether the substrates were soaked in PBS or biofilm.

### Example 22

### Staining Studies on HAP Substrates

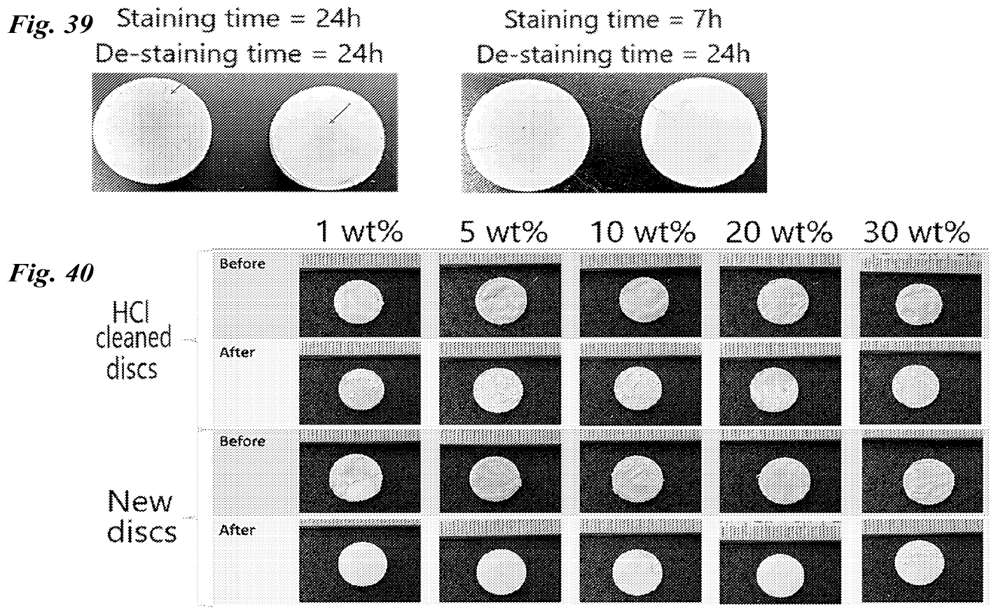

To improve the contrast for ImageJ analysis on HAP substrates, a Coomassie stain (mixture of water, acetic acid, methanol and Coomassie Blue) was used. It was found that staying the substrates for 7 hours and then de-staining them using a detaining solution (mixture of water, acetic acid, and methanol) for 24 hours resulted in the best contrast in control samples as shown in FIG. 39.

To be able to reuse HAP discs, the pre-used discs were soaked in 12M HCl for 5 minutes to clean the surface. Hydrochloric acid (HCl) dissolves HAP, which caused the surface of the discs to become rougher due to etching by HCl. The discs were immersed in simulated saliva for 5 minutes. The excess saliva was removed from disc surface after which Z40T7 and Z26T21 were applied to the discs. The discs were then allowed to cure in saliva for 10 minutes. As shown in FIG. 40, the discs cleaned by HCl had good adhesion to the zein adhesives, while the brand new discs with a smoother surface showed poor adhesion.

### Example 23

### Adhesion Studies on Limestone (Effect of Curing in Air Vs Under Saliva)

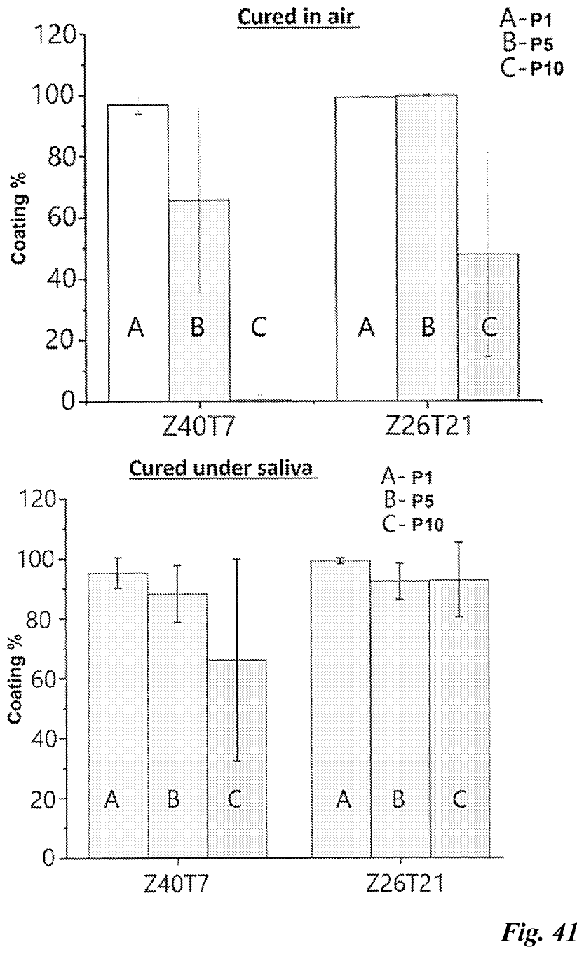

Adhesion of adhesive coatings was also studied on limestone substrates. The substrates were immersed in simulated saliva for 5 minutes prior to adhesive application. 20 μl of the adhesive was coated onto the substrates using a spatula. The adhesive was allowed to cure for 10 minutes either in air or under simulated saliva. A commercial water flosser (Waterpik Ultra) was rastered on the coatings for 1 minute to study the adhesion of coatings. The water flosser tip was set at a constant height of 1 mm from the substrate. The images of the substrate before and after the water flosser tests were captured to analyze the amount of adhesive remaining on the substrates after the test. FIG. 41 shows the percentage of coating remaining after water flosser tests at different pressure settings. It was observed that the adhesive retention was better for Z26T21 after water flosser experiments even at high pressures. Moreover, the adhesion was even better when the substrates were cured under saliva instead of in air.

### Example 24

### Adhesion Studies on Hydroxyapatite Coated Glass

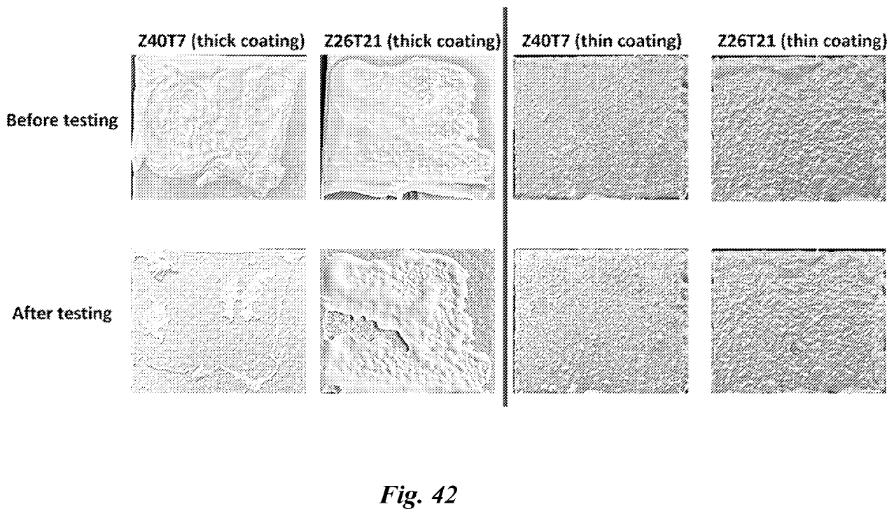

Adhesion of adhesive coatings was also studied on hydroxyapatite coated glass substrates. The coatings were cured under saliva for 10 minutes and testing was performed at pressure 5. When the adhesive was applied as a thicker layer, the coating retention was poor but when the adhesive was applied as a thinner layer, the coatings retained much better as shown in FIG. 42.

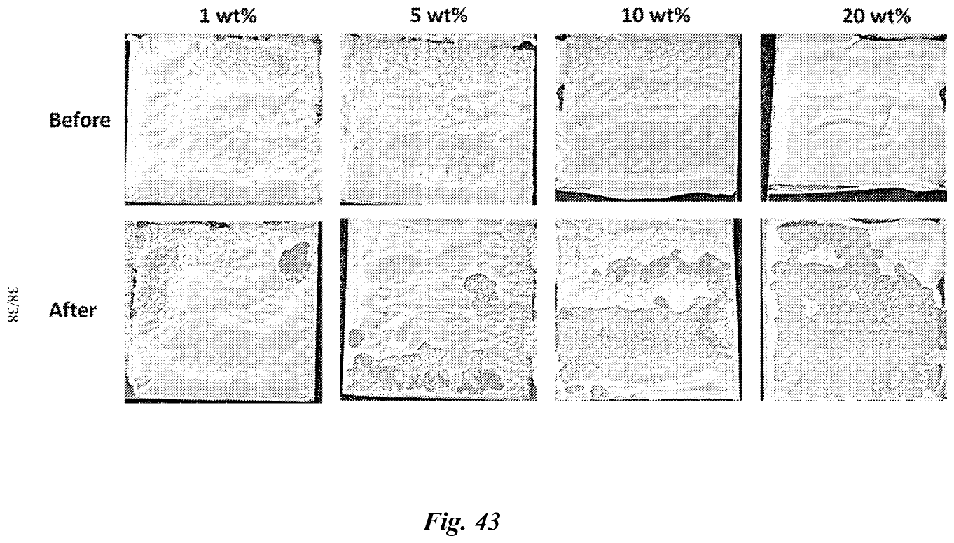

The effect of incorporating hydroxyapatite powder as a filler in Z26T21 was also evaluated. The filler content varied from 1 to 20 wt. %. As shown in FIG. 43, the adhesion was better at lower filler content. The coatings failed cohesively at higher filler content.

1. An adhesive comprising:
zein and one or more catechol- or gallol-containing compounds; or
zein, one or more catechol- or gallol-containing compounds, and an interfacial crosslinker, an iron crosslinker, or both an interfacial crosslinker and an iron crosslinker.

2. (canceled)

3. The adhesive of claim 1, wherein the one or more catechol- or gallol-containing compound comprises catechol, vanillin, caffeic acid, juglone (5-hydroxy-1,4-naphthoquinone), catecholamine, resveratrol, 3,4-dihyroxyphenylacetic acid (DOPAC), catechin hydrate, 3,4-dihydroxy benzoic acid, 3,4-dihydroxybenzaldehyde, tannic acid (TA), gallic acid, gallotannins, ellagic acid, eugallol, 1,2,3-trihydroxybenzene, quercetin, gallol, or any combination thereof.

4. The adhesive of claim 1 formulated at a pH of at or about 6-8, the one or more catechol- or gallol-containing compound is tannic acid, and/or wherein the zein is present in at or about 25-45 weight percent of the adhesive prior to curing.

5. (canceled)

6. The adhesive of claim 1, wherein the adhesive comprises an interfacial crosslinker comprising transglutaminase (TGnase), ethyl-3-(3-dimethylaminopropyl)carbodiimide hydrochloride (EDC), N-hydroxy succinimide (NHS), an elastin-like polypeptide (ELP), polyethylene glycol (PEG), a dendrimer, glutaraldehyde, and/or a photocrosslinker.

7. (canceled)

8. The adhesive of claim 3, wherein zein is at or about 25-45 weight percent and the one or more catechol- or gallol-containing compound is tannic acid present in at or about 5-30 weight percent of the uncured adhesive.

9-13. (canceled)

14. The adhesive of claim 1, further comprising a solvent that is an aqueous solvent, an organic solvent, or both an aqueous and organic solvent.

15. (canceled)

16. The adhesive of claim 14 wherein the solvent comprises ethanol and water present by volume in at or about a ratio of (2-5):1.

17. The adhesive of claim 1 having a tensile strength of up to about 20 MPa.

18. The adhesive of claim 1 further comprising at least one filler.

19. The adhesive of claim 18, wherein the at least one filler comprises hydroxyapatite, calcium carbonate, calcium phosphate, magnesium phosphate, laponite, montmorillonite, or a combination of any of the foregoing.

20. (canceled)

21. A tissue sealant comprising a zein, tannic acid, and a solvent.

22-23. (canceled)

24. The tissue sealant of claim 21, consisting of zein, tannic acid, ethanol and water.

25. The tissue sealant of claim 21, further comprising an interfacial crosslinker, an iron crosslinker, or both an interfacial crosslinker and an iron crosslinker.

26-28. (canceled)

29. A method of sealing a damaged tissue of a subject comprising applying an adhesive or a tissue sealant to a damaged tissue, wherein
the adhesive comprises (a) zein and one or more catechol- or gallol-containing compounds, or (b) zein, one or more catechol- or gallol-containing compounds, and an interfacial crosslinker, an iron crosslinker, or both an interfacial crosslinker and an iron crosslinker; and
the tissue sealant comprises a zein, tannic acid, and a solvent.

30. The method of claim 29, wherein the method further comprises:
applying an interfacial crosslinker to the damaged tissue to form a first layer, and
applying the adhesive or tissue sealant to the first layer.

31-33. (canceled)

34. The method of claim 29, wherein the damaged tissue is selected from the group consisting of skin, heart tissue, stomach tissue, lung tissue, liver tissue, intestines, dura mater, and an aorta, or the damaged tissue is in an oral environment.

35-51. (canceled)

52. A method of formulating an adhesive comprising:
forming a first solution by combining zein, one or more catechol- or gallol-containing compounds, a solvent, and, optionally, an interfacial crosslinker, an iron crosslinker, or both an iron crosslinker and an interfacial crosslinker;
adjusting a pH of the first solution to at or about 6-8 if needed; and
curing the first solution to substantially remove the solvent therefrom.

53. The method of claim 52, wherein curing the first solution comprises incubating the first solution at between about 31.0° C. to about 42.0° C. and/or curing the first solution comprises light curing and/or exposing the first solution to oxygen.

54-56. (canceled)

57. The method of claim 52, wherein the first solution further comprises TGnase.

58. The method of claim 52, further comprising applying the cured first solution to a layer comprising TGnase.

59. (canceled)

61. The method of claim 52, wherein the solvent comprises ethanol and water, and an ethanol to water ratio of the solvent is about 5:1, about 4:1, about 3:1, or about 2:1, and/or
the ratios of the amount of zein, the one or more catechol- or gallol-containing compound, and the solvent are adjusted to tune one or more characteristics of the adhesive to correspond with mechanical properties of a targeted tissue.

62-70. (canceled)
